(19) United States
(12) Patent Application Publication
(10) Pub. No.: US 2023/0265508 A1
(43) Pub. Date: Aug. 24, 2023

(54) PREFERENTIAL GENERATION OF iPSC CARRYING ANTIGEN SPECIFIC TCRs FROM TUMOR INFILTRATING LYMPHOCYTES

(71) Applicant: The United States of America,as represented by the Secretary,Department of Health and Human Services, Bethesda, MD (US)

(72) Inventors: Raul E. Vizcardo, Foster City, CA (US); SM Rafiqul Islam, Bethesda, MD (US); Naritaka Tamaoki, South San Francisco, CA (US); Takuya Maeda, Bethesda, MD (US); Nicholas P. Restifo, Chevy Chase, MD (US)

(73) Assignee: The United States of America,as represented by the Secretary,Department of Health and Human Services, Bethesda, MD (US)

(21) Appl. No.: 18/020,823

(22) PCT Filed: Aug. 20, 2021
(86) PCT No.: PCT/US2021/046969

§ 371 Date: Feb. 10, 2023

Related U.S. Application Data
(60) Provisional application No. 63/068,458, filed on Aug. 21, 2020.

(51) Int. Cl. C12Q 1/6881; C12N 5/074

(52) U.S. Cl. CPC C12Q 1/6881; C12N 5/0696; C12N 2506/11; C12N 2501/515; C12N 2501/51; C12N 2502/30

(57) ABSTRACT

Disclosed are methods for reprogramming cancer-reactive T cells into iPSC cells as well as methods utilizing such cells for the identification of cancer-antigen specific TCRs and the treatment of cancer.

### CROSS-REFERENCE TO RELATED APPLICATION

This patent application claims the benefit of co-pending U.S. Provisional Patent Application No. 63/068,458 filed Aug. 21, 2020, which is incorporated by reference in its entirety herein.

### STATEMENT REGARDING FEDERALLY SPONSORED RESEARCH OR DEVELOPMENT

This invention was made with Government support under project number Z01ZIA BC0100763 by the National Institutes of Health, National Cancer Institute. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 32,662 Byte ASCII (Text) file named “755124_ST25.txt,” created on Aug. 20, 2021.

### BACKGROUND OF THE INVENTION

T cells, such as tumor infiltrating lymphocytes, can be grown, expanded ex vivo, and may be infused into subjects to treat cancers such as, e.g., melanoma, cervical cancer, gastrointestinal (GI) cancers, and breast cancer. Examples of such treatment methods include adoptive cell therapy (ACT). However, it can be difficult to identify cancer-specific TCRs from tumor-infiltrating lymphocytes (TILs) due to the subject-specific nature of many cancer antigens (e.g., neoantigens).

Further, upon chronic exposure to cancer antigens, T cells, e.g., TILs, may become terminally differentiated, senescent and anergic. Therefore, the therapeutic efficacy of such cells may be attenuated. The rejuvenation of TILs via induced pluripotent stem cell (iPSC) reprogramming can address these limitations. However, conventional methods for generating cancer antigen-specific iPSCs from T cell populations may be inefficient. Such methods can involve the activation of the T cell receptor (TCR) by anti-CD3 and/or anti-CD28 antibodies to stochastically reprogram T cells into iPSCs carrying inherited TCRs. However, such broad TCR activation may reprogram T cells even if they do not carry a cancer antigen-specific TCR. Therefore, conventional methods for reprogramming T cells typically start with a T cell line carrying a monoclonal TCR that is pre-expanded and pre-screened for cancer antigen specificity. As such, these conventional methods may be difficult, inefficient, time-consuming and may limit the diversity of generated cancer antigen-specific TCRs.

Accordingly, there is a need for improved methods suitable for producing previously unidentified cancer antigen-specific iPSC lines, as well as methods that may be used to identify novel cancer antigen-specific TCRs.

### BRIEF SUMMARY OF THE INVENTION

Aspects of the present disclosure include methods for producing an isolated population of tumor antigen specific T-cell induced pluripotent stem cells (T-iPSCs), comprising:
(a) isolating T cells from a first sample from a subject, wherein said subject has cancer;
(b) contacting the isolated T-cells of (a) with one or more tumor antigens to produce co-cultured T-cells;
(c) isolating from the co-cultured T-cells, T-cells expressing one or more T cell activation markers; and
(d) contacting the isolated T-cells of (c) with one or more reprogramming factors under conditions sufficient to reprogram the cells into T-iPSCs.

Aspects of the present disclosure also include methods for treating or preventing cancer, comprising:
(a) producing an isolated population of iPSCs according to methods described herein with respect to other aspects of the invention;
(b) differentiating the iPSCs into T lineage cells, to obtain differentiated T lineage cells; and
(c) administering the differentiated T lineage cells to the subject in an amount effective to treat or prevent cancer in the subject.

Aspects of the present disclosure also include methods for producing a medicament for the treatment or prevention of cancer in a subject having cancer, comprising:
(a) producing an isolated population of iPSCs according to methods described herein with respect to other aspects of the invention;
(b) differentiating the iPSCs into T lineage cells, to obtain differentiated T lineage cells; and
(c) formulating the differentiated T lineage cells into a medicament for the treatment or prevention of cancer in the subject.

Aspects of the present disclosure also include methods for identifying a cancer antigen-specific TCR, comprising:
(a) producing an isolated population of iPSCs according to methods described herein with respect to other aspects of the invention; and
(b) determining the nucleotide sequence encoding the cancer antigen-specific TCR by performing RNA or DNA sequencing of nucleic acids comprised in the isolated population of iPSCs.

Aspects of the present disclosure also include methods for identifying a tumor antigen specific TCR, comprising:
(a) isolating T cells from a first sample from the subject, wherein said subject has cancer;
(b) contacting the isolated T-cells of (a) with one or more tumor antigens to produce co-cultured T-cells;
(c) isolating from the co-cultured T-cells of (b) T-cells expressing one or more T cell activation markers;
(d) contacting the isolated T-cells of (c) with one or more reprogramming factors under conditions sufficient to reprogram the cells into T-iPSCs; and
(e) determining the DNA sequence encoding the TCR alpha and TCR beta chain from an iPSC colony.

Aspects of the present disclosure also include methods for generating a polyclonal population of tumor antigen specific iPSC derived T cells, comprising:
(a) isolating T cells from a first sample from the subject, wherein said subject has cancer;
(b) contacting the isolated T-cells of (a) with one or more tumor antigens to produce co-cultured T-cells;
(c) isolating from the co-cultured T-cells of (b) T-cells expressing one or more T cell activation markers;
(d) contacting the isolated T-cells of (c) with one or more reprogramming factors under conditions sufficient to reprogram the cells into T-iPSCs; and
(e) differentiating the iPSCs into T lineage cells, to obtain differentiated T lineage cells.

Aspects of the present disclosure also include a recombinant TCR comprising:
(a) a CDR3 Va region of any one of SEQ ID Nos 1-4 and a CDR3 Vb region of any one of SEQ ID NOs. 5-8;
(b) A V alpha chain of SEQ ID NO:9 and a V beta chain of SEQ ID NO:13;
(c) A V alpha chain of SEQ ID NO:10 and a V beta chain of SEQ ID NO:14;
(d) V alpha chain of SEQ ID NO:11 and a V beta chain of SEQ ID NO:15; or
(e) V alpha chain of SEQ ID NO:12 and a V beta chain of SEQ ID NO:16.

Aspects of the present disclosure also include a chimeric TCR comprising:
(a) CDR3 Va region of any one of SEQ ID Nos 1-4 and a CDR3 Vb region of any one of SEQ ID NOs. 5-8;
(b) A V alpha chain of SEQ ID NO:9 and a V beta chain of SEQ ID NO:13;
(c) A V alpha chain of SEQ ID NO:10 and a V beta chain of SEQ ID NO:14;
(d) V alpha chain of SEQ ID NO:11 and a V beta chain of SEQ ID NO:15; or
(e) V alpha chain of SEQ ID NO:12 and a V beta chain of SEQ ID NO:16.

Aspects of the present disclosure also include an isolated cell expressing a recombinant TCR or a chimeric TCR according to other aspects of the disclosure.

Aspects of the present invention also include compositions comprising iPSCs produced according to methods described herein.

Aspects of the present invention also include compositions comprising differentiated T lineage cells produced according to methods described herein.

### BRIEF DESCRIPTION OF THE SEVERAL VIEWS OF THE DRAWING(S)

FIG. 1 is a bar graph indicating the number of alkaline phosphatase (AP) positive colonies derived from 1×10⁵ T cells with or without TCR stimulation. N=3 for each donor.

FIG. 2A is a diagram providing a schematic representation of a process via which TILs are sorted and reprogramed into iPSCs. To briefly summarize, an autologous tumor cell line and a TIL fragment culture were co-cultured for 16 hours, sorted for CD3⁺ CD4⁻ CD8⁺ PD1⁺ 4-1BB⁺ TIL, and transduced with sendai virus containing 4 Yamanaka factors and the SV40 large T antigen. On day 20-21 when cells formed domed shaped ES-like colonies, they were picked up as clones.

FIG. 2B is a graph indicating the frequency of pre-identified tumor reactive TCRs in starting cells (Bulk) and in sorted PD1⁺ 4-1BB⁺ (DP) cells.

FIG. 2C is a graph depicting enrichment analysis (the ratio of DP/Bulk) of those six pre identified TCRs.

FIG. 3A is a series of graphs depicting the FACS gating strategy used to sort CD3⁺ CD8⁺ PD1⁺ 4-1BB⁺ TILs (from patient 3784) after tumor-TIL co-culture. The upper panel shows the phenotype of TIL without tumor cell co-culture. Dump gate is mixture of different lineage markers, to exclude T_{reg} cells (CD25), NK cells (CD56), and gamma delta T cells.

FIG. 3B is the first in a series of five bar graphs (FIGS. 3B-3F) showing the relative frequency of TCR clones. The TCRs were present in the bulk population (i.e. the starting cells) and were reprogrammed to iPSCs, in four sorted populations (PD1⁻ 4-1BB⁻, PD1⁺ 4-1BB⁻, PD1⁻ 4-1BB⁺, PD1⁺ 4-1BB⁺) after TIL-tumor cell co-culture. The frequency is presented relative to bulk frequency of each TCR clone before co-culture.

FIG. 3C is the second in a series of five bar graphs (FIGS. 3B-3F) showing the relative frequency of TCR clones. The TCRs were present in the bulk population (i.e. the starting cells) and were reprogrammed to iPSCs, in four sorted populations (PD1⁻ 4-1BB⁻, PD1⁺ 4-1BB⁻, PD1⁻ 4-1BB⁺, PD1⁺ 4-1BB⁺) after TIL-tumor cell co-culture. The frequency is presented relative to bulk frequency of each TCR clone before co-culture.

FIG. 3D is the third in a series of five bar graphs (FIGS. 3B-3F) showing the relative frequency of TCR clones. The TCRs were present in the bulk population (i.e. the starting cells) and were reprogrammed to iPSCs, in four sorted populations (PD1⁻ 4-1BB⁻, PD1⁺ 4-1BB⁻, PD1⁻ 4-1BB⁺, PD1⁺ 4-1BB⁺) after TIL-tumor cell co-culture. The frequency is presented relative to bulk frequency of each TCR clone before co-culture.

FIG. 3E is the fourth in a series of five bar graphs (FIGS. 3B-3F) showing the relative frequency of TCR clones. The TCRs were present in the bulk population (i.e. the starting cells) and were reprogrammed to iPSCs, in four sorted populations (PD1⁻ 4-1BB⁻, PD1⁺ 4-1BB⁻, PD1⁻ 4-1BB⁺, PD1⁺ 4-1BB⁺) after TIL-tumor cell co-culture. The frequency is presented relative to bulk frequency of each TCR clone before co-culture.

FIG. 3F is the fifth in a series of five bar graphs (FIGS. 3B-3F)h showing the relative frequency of TCR clones. The TCRs were present in the bulk population (i.e. the starting cells) and were reprogrammed to iPSCs, in four sorted populations (PD1⁻ 4-1BB⁻, PD1⁺ 4-1BB⁻, PD1⁻ 4-1BB⁺, PD1⁺ 4-1BB⁺) after TIL-tumor cell co-culture. The frequency is presented relative to bulk frequency of each TCR clone before co-culture.

FIG. 4A is a series of graphs presenting representative FACS plots of various candidate TCR transduced T cells co-cultured with tumor cells for 16 hours.

FIG. 4B is a bar graph showing the percentage of 4-1BB+ cells in various conditions. Representative data of four independent experiments are presented: Control PBL; no transgene, GFP-PBL; GFP transduced, TIL; expanded TIL containing tumor reactive T cells.

FIG. 4C is a graph summarizing an ELISA IFNg production assay of T cells transduced with candidate TCR alpha and beta pairs identified from TIL-iPSCs, and co-cultured with tumor cells for 16 hours.

FIG. 5A is a diagram depicting the reprogramming process for healthy donor Peripheral Blood Mononuclear Cells (PBMCs): T cells were separated, stimulated by anti-CD3 and anti-CD28 beads, and transduced with Sendai virus containing 4 Yamanaka factors and SV40 large T antigen.

FIG. 5B is a series of representative FACS plots depicting the strategy for sorting naïve (CD62L+CCR7+CD45RA+), central memory (CD62L+CD45RO+CCR7+), effector memory (CD62L−CD45RO+CCR7−CD45RA−) and EMRA (CD62L−CD45RO−CCR7−CD45RA+) cell populations from healthy donor PBMCs.

FIG. 5C is a series of representative FACS plots depicting post-sort gating to check the purity of each of the following cell populations: naïve (CD62L+CD45RO−CCR7+CD45RA+), central memory (CD62L+CD45RO+CCR7+CD45RA−), effector memory (CD62L−CD45RO+CCR7−CD45RA−) and EMRA (CD62L−CD45RO−CCR7−CD45RA+).

FIG. 6 is a diagram providing a schematic representation of TCR sequencing of TIL-iPSCs and reactivity testing. Briefly, from each dish of TIL-iPSC colonies as many clones as possible were picked up, placed into each well of culture plates, expanded, passaged up to three times and frozen down for further experiments. Before freezing about 15-20 individual iPSC lines, about 100,000 cells each, were pooled together into a master tube and genomic DNA was extracted for TCR beta immunoseq analysis. After picking up the colonies, all the remaining iPSC colonies from the mother tube (approximately 600-900 iPSC colonies) were also collected in a tube for genomic DNA extraction and TCR beta immunoseq analysis. To test the reactivity of TCRs identified in TIL-iPSC master tubes, individual iPSCs were re-submitted for TCR alpha and beta sequencing, cloning into gamma retrovirus vector, transduced into PBL of healthy volunteers, and tested for reactivity against patient's tumor cells via a 4-1BB upregulation assay and a cytokine production assay by ELISA.

FIG. 7A is the first in a series of three figures (FIGS. 7A-7C) depicting series of plots illustrating the FACS gating strategy to sort CD3+ CD4−CD8+PD1+ 4-1BB+ TIL after tumor-TIL co-culture from patient 1913. The upper panels show the phenotype of TIL without tumor cell co-culture. The middle panels indicate phenotypic expression of PD1+ 4-1BB+ cells after 16 hrs co-culture with autologous tumor cells. The bottom panels indicate post-sort purity of PD1+ 4-1BB+ cells.

FIG. 7B the second in a series of three figures (FIGS. 7A-7C) depicting a series of plots illustrating the FACS gating strategy to sort CD3+ CD4− CD8+ PD1+ 4-1BB+ TIL after tumor-TIL co-culture from patient 1913. The upper panels show the phenotype of TIL without tumor cell co-culture. The middle panels indicate phenotypic expression of PD1+ 4-1BB+ cells after 16 hrs co-culture with autologous tumor cells. The bottom panels indicate post-sort purity of PD1+ 4-1BB+ cells.

FIG. 7C the third in a series of three figures (FIGS. 7A-7C) depicting series of plots illustrating the FACS gating strategy to sort CD3+ CD4−CD8+ PD1+ 4-1BB+ TIL after tumor-TIL co-culture from patient 1913. The upper panels show the phenotype of TIL without tumor cell co-culture. The middle panels indicate phenotypic expression of PD1+ 4-1BB+ cells after 16 hrs co-culture with autologous tumor cells. The bottom panels indicate post-sort purity of PD1+ 4-1BB+ cells.

FIG. 7D is a bar graph depicting enrichment (the ratio of DP/Bulk) of the 9 different TCRs in post-sorted PD1+ 4-1BB+ cells from patient 1913.

### DETAILED DESCRIPTION OF THE INVENTION

Disclosed herein are methods for selectively reprogramming antigen-specific T cells. In various embodiments, the T cells for reprogramming are heterogeneous TIL populations isolated from a tumor. In aspects of the present disclosure, it was found that TCR stimulation through, for example, by contact (by co-culture) with tumor antigen, followed by exposure to reprogramming factors (e.g., four Yamanaka factors+SV40), resulted in the preferential reprogramming of tumor reactive T cells from heterogeneous populations of T cells (e.g., from populations of TILs). In various embodiments, the present disclosure provides for enriching the population of activated T cells after co-culturing with a tumor antigen by selecting for T cells expressing one or more activation markers (e.g., 4-1BB and/or PD-1) before being reprogrammed into iPSC cells using reprogramming factors. Such methods have been demonstrated to achieve a heterogeneous population T cell derived IPSCs (T-IPSCs) with TCRs reactive to a specific tumor antigen. In various embodiments, the present disclosure provides for methods of developing novel anti-tumor T cell therapies. In various embodiments, such methods may be used to identify novel antigen-specific T cells and TCRs including extremely rare tumor antigen specific TCRs, which may be used to construct novel engineered TCRs for use in adoptive T cell therapy (ACT). Alternatively, in various aspects of the present disclosure, the heterogeneous population of T-iPSCs can be differentiated into T cells expressing tumor antigen-specific TCRs.

### I. Isolated T Cells for Reprogramming to T-Cell Induced Pluripotent Stem Cells (T-iPSCs)

In various embodiments, a method is provided for producing an isolated population of tumor antigen specific T-cell induced pluripotent stem cells (T-iPSCs). As used herein, a “T-cell induced pluripotent stem cell” or “T-iPSC” is an induced pluripotent stem cell that has been generated from an antigen specific T cell and that has retained the V(D)J recombination of the TCR gene, or otherwise retains the TCR specificity of the original isolated T cell.

In various embodiments, the T-iPSC is generated from an isolated T cell taken from a sample from a subject, wherein said subject has cancer. The term “isolated,” as used herein, means having been removed from its natural environment, for example in the case of non-nonreactive/bystander tumor reactive T cells. The term “purified,” as used herein, means having been increased in purity, wherein “purity” is a relative term, and not to be necessarily construed as absolute purity. For example, the purity can be at least about 50%, can be greater than about 60%, about 70% or about 80%, about 90% or can be about 100%.

Aspects of the disclosure may generally comprise providing a sample comprising T cells from a subject having cancer. Any biological sample comprising T cells can be used. In aspects, the biological sample is a tumor sample or a sample of peripheral blood. Examples of biological samples that may be used in accordance with the disclosure include, without limitation, tissue from primary tumors, tissue from the site of metastatic tumors, exudates, effusions, ascites, fractionated peripheral blood cells, bone marrow, peripheral blood buffy coat, and cerebrospinal fluid. In aspects, the sample may be obtained by fragment culture. As such, the biological sample may be obtained by any suitable means, including, without limitation, aspiration, biopsy, resection, venous puncture, arterial puncture, lumbar spinal puncture, shunts, catheterization, blood draw, leukapheresis, or the placement of a drain.

In various embodiments, the present disclosure relates to isolating T cells from a source and reprogramming said T cells. Examples of suitable source cells include, but are not limited to, peripheral blood mononuclear cells (PBMCs). T cells for use in the methods herein may include, but are not limited to, cultured T cells, e.g., primary T cells or T cells from a cultured T cell line, e.g., Jurkat, SupT1, etc., or T cells obtained from a mammal. If obtained from a mammal, the source cells can be obtained from numerous sources, including but not limited to blood, bone marrow, lymph node, tumor, thymus, spleen, or other tissues or fluids. Source cells can also be enriched for or purified. The T cells can be any type of T cells and can be of any developmental stage, including but not limited to CD4+CD8αβ+ double positive T cells, CD4+ helper T cells, e.g., Th1 and Th2 cells, CD4+ T cells, CD8+ T cells (e.g., cytotoxic T cells), peripheral blood mononuclear cells (PBMCs), peripheral blood leukocytes (PBLs), tumor infiltrating cells (TILs), memory T cells, naïve T cells, and the like.

In one embodiment, the T cells for use in the reprogramming methods described herein are isolated from TILs. By “tumor infiltrating lymphocytes” or “TILs” herein is meant a population of cells originally obtained as white blood cells that have left the bloodstream of a subject and migrated into a tumor. TILs include, but are not limited to, CD8+ cytotoxic T cells (lymphocytes), Th1 and Th17 CD4+ T cells, natural killer cells, dendritic cells and M1 macrophages. TILs include both primary and secondary TILs. “Primary TILs” are those that are obtained from patient tissue samples as outlined herein (sometimes referred to as “freshly harvested”). In some embodiments, TILs can generally be categorized by expressing one or more of the following biomarkers: CD4, CD8, TCR ab, CD27, CD28, CD56, CCR7, CD45Ra, CD95, PD-1, and CD25.

Aspects of the disclosure may generally comprise dissociating a tumor sample into a suspension comprising T cells. In aspects, the tumor cells may be obtained by fragment culture. The dissociation of the tumor sample may be carried out for a time and under conditions sufficient to obtain a suspension of cells normally found in a tumor including, for example, fibroblasts, tumor cells, T cells, and many other types of cells. Dissociating the tumor sample may be carried out in any of a variety of different ways known in the art. For example, the tumor sample may be dissociated enzymatically, mechanically, by hand (e.g., sharp dissection), or a combination of any of the foregoing. Mechanical dissociation may be carried out using, for example, a GENTLEMACS dissociator (Miltenyi Biotec, Auburn, Calif.).

Aspects of the present disclosure include providing samples from a subject. Unless stated otherwise, as used herein, the term “subject” refers to any mammal including, but not limited to, mammals of the order Lagomorpha, such as rabbits; the order Carnivora, including Felines (cats) and Canines (dogs); the order Artiodactyla, including Bovines (cows) and Swines (pigs); or of the order Perssodactyla, including Equines (horses). In aspects, the mammals are non-human primates, e.g., of the order Primates, Ceboids, or Simoids (monkeys) or of the order Anthropoids (humans and apes). In some aspects, the mammal may be a mammal of the order Rodentia, such as mice and hamsters. In aspects, the mammal is a non-human primate or a human. In other aspects, the subject is a human.

Aspects of the disclosure include providing a sample from a subject with cancer and/or a tumor. In aspects, the cancer comprises cancer cells. In aspects, the cancer cells comprise tumor cells. The cancer may be any cancer, including, e.g., acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, cholangiocarcinoma, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In aspects, the cancer is melanoma, cervical cancer, GI cancer, or breast cancer. In aspects, the cancer is melanoma.

In aspects, the subject may have a tumor, e.g. a solid tumor. The tumor may be a mass resulting from abnormal, excessive growth of tissue in the subject. The tumor may comprise tumor cells. The tumor may be potentially malignant, or malignant (i.e. cancerous). The tumor may be a primary tumor or a metastatic tumor. In aspects, the tumor is associated with cancer, e.g., any of the cancers described herein. In such aspects, the tumor will comprise tumor cells that are also referred to herein as cancer cells. In aspects, the tumor is associated with melanoma, cervical cancer, GI cancer, or breast cancer. In aspects, the tumor is associated with melanoma.

Aspects of the disclosure include providing a sample from a subject comprising T cells. As used herein, the T cell may be, for example, a human T cell. The T cell can be any type of T cell and can be of any developmental stage, including but not limited to, CD4⁺/CD8⁺ double positive T cells, CD4⁺ T cells, e.g., Th₁ and Th₂ cells, CD8⁺ T cells (e.g., cytotoxic T cells), Th₉ cells, TIL, memory T cells, naïve T cells, and the like. The T cell may be a CD8⁺ T cell or a CD4⁺ T cell. Naïve T cells are mature T cells that have not encountered a cognate antigen within their periphery. Naïve T cells are commonly characterized by the surface expression of L-selectin (CD62L) and C—C Chemokine receptor type 7 (CCR7), the absence of the activation markers CD25, PD-1 or CD69, the absence of memory CD45RO isoform, and/or expression of functional IL-7 receptors, including subunits IL-7 receptor-α, CD127, and common-7 chain, CD132. In an aspect, the T cells are tumor-infiltrating lymphocytes (TILs).

According to the present disclosure, T cells may comprise at least one T cell receptor or “TCR”. A TCR is a protein complex found on the surface of T cells or T lymphocytes that recognizes and binds an antigen, e.g. a cancer antigen in the context of MHC. The T cells can comprise about 1, about 2, about 3, about 4, about 5, about 6, about 7, about 8, about 9, or about 10 TCRs, or any range of numbers of the foregoing (e.g., about 1 to about 10, about 2 to about 10, about 1 to about 9, about 2 to about 9, etc.). A TCR generally comprises two polypeptides (i.e., polypeptide chains), such as an alpha-chain of a TCR, a beta-chain of a TCR, a γ-chain of a TCR, a δ-chain of a TCR, or a combination thereof. Such polypeptide chains of TCRs are known in the art. The antigen-specific TCR can comprise any amino acid sequences, provided that the TCR can specifically bind to and immunologically recognize an antigen of interest, such as, e.g., a cancer antigen, a tumor antigen, or an epitope thereof. In certain aspects of the disclosure, the TCR may be a cancer antigen-specific TCR. In aspects the TCR may be tumor antigen-specific TCR.

In aspects, the present disclosure includes providing a sample containing T cells from the subject having cancer and/or a tumor. The sample can be any suitable sample (liquid or solid) that has T cells present in a sufficient quantity.

The method may further comprise isolating the T cells from the other cells of the sample that are not T cells to produce isolated T cells and an isolated population of cells that are not T cells. This separation step may be accomplished using any suitable technique known to those in the field, for example, fluorescence-activated cell sorting (FACS), magnetic separation (MACs), acoustic separation, and electrokinetic separation.

II. Co-Culturing Isolated T Cells with a Tumor Antigen

In various embodiments of the present disclosure, the isolated T cells are contacted with one or more tumor antigens to produce co-cultured T-cells. As used herein, the term “co-cultured T-cells” refers to T-cells, which have been cultured in vitro in the presence of at least one tumor antigen for a period of time. In various embodiments, this co-culturing step when followed by reprogramming has been shown to preferentially reprogram tumor reactive T cells from heterogeneous populations of T cells.

The co-culturing may be carried out for a suitable duration of time. In aspects, the co-culturing may proceed for about 3 to 72 hours. In certain aspects, the co-culturing may proceed for about 3 to 48 hours. In aspects, the co-culturing may proceed for about 8-48 hours. In aspects, the co-culturing may proceed for about 8-24 hours. In aspects, the co-culturing may proceed for about 12-20 hours. In aspects, the co-culturing may proceed for about 16 hours.

The term “tumor antigen” as used herein, refers to any molecule (e.g., protein, polypeptide, peptide, lipid, carbohydrate, etc.) solely or predominantly expressed or over-expressed by a tumor cell or cancer cell, such that the antigen is associated with the tumor or cancer. In various embodiments, the tumor antigen is a cancer antigen. The cancer antigen can additionally be expressed by normal, non-tumor, or non-cancerous cells. However, in such cases, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor or non-cancerous cells. Also, the cancer antigen can additionally be expressed by cells of a different state of development or maturation. For instance, the cancer antigen can be additionally be expressed by cells of the embryonic or fetal stage, which cells are not normally found in an adult host. Alternatively, the cancer antigen can be additionally expressed by stem cells or precursor cells, which cells are not normally found in an adult host. In an embodiment, the TCR alpha-chain and beta-chain pair has specificity for a melanoma antigen.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor or non-cancer cells. Cancer antigens are known in the art and include, for instance, CXorf61, mesothelin, CD19, CD22, CD276(B7H3), gp100, MART-1, Epidermal Growth Factor Receptor Variant III (EGFRVIII), TRP-1, TRP-2, tyrosinase, NY-ESO-1 (also known as CAG-3), MAE-1, MAGE-3, etc.

In aspects of the disclosure, the cancer/tumor antigen is a cancer/tumor neoantigen. A neoantigen is an immunogenic mutated amino acid sequence that is encoded by a cancer-specific mutation. Neoantigens are not expressed by normal, non-cancerous cells and may be unique to the subject. ACT with T cells which have antigenic specificity for a neoantigen may provide a “personalized” therapy for the subject.

In various embodiments, the isolated T cells are co-cultured with peptide pools, wherein the peptide pools comprise a tumor antigen. Peptide pools are routinely used in the art to stimulate antigen-specific T cell populations and to study T cell responses. See, e.g., Suneetha et al., J Immunol Methods 2009, 342(1-2):33-48. In various embodiments, the T-cells are co-cultured in the presence of a biological sample capable of expressing a tumor antigen. In various embodiments, the biological sample is isolated from the same subject having cancer, and/or a tumor. In various embodiments, the isolated T cells are co-cultured with cancer cells expressing said tumor antigen. In various embodiments, the isolated T cells are co-cultured with autologous tumor cells. In various embodiments, the isolated T cells are co-cultured with irradiated tumor cells. In various embodiments, the isolated T cells are co-cultured with a tumor cell line.

Any biological sample comprising cancer cells can be used, for example, any of the biological samples described herein with respect to other aspects of the invention. The biological sample can be obtained by any suitable method known to those in the field. Such methods include, without limitation, those described herein with respect to other aspects of the invention.

According to aspects of the present disclosure, cancer cells in the second sample may be isolated to produce isolated cancer cells. Methods for isolating the cancer cells are known in the art. This separation step may be accomplished using any suitable technique known to those in the field, for example, FACS, magnetic separation (MACs), acoustic separation, and electrokinetic separation.

In aspects of the present disclosure, the isolated cancer cells and/or tumor cells are co-cultured with the isolated T-cells. Appropriate cell culture techniques are well known to those skilled in the art. Co-culturing the T cells with their autologous isolated cancer/tumor cells may comprise, for example, culturing the cells in any suitable cell culture media. Examples of cell culture media that may be useful in the disclosed methods include those that are typically used for culturing T cells and may include, e.g., Roswell Park Memorial Institute (RPMI) media+10% fetal bovine serum (FBS), and RPMI+10% human AB serum. The cell culture media may further comprise any of a variety of additives. For example, the cell culture medium may further comprise one or more antibodies and/or one or more cytokines. In aspects, cell culturing may proceed in the absence of cytokines. Cell culturing may be carried out in any suitable vessel or vessels. For example, cell culturing may be carried out in multi-well plates, e.g., 24-well plates, 96-well plates or 384-well plates.

In aspects, co-culturing the T-cells with the cancer cells and/or tumor cells will produce co-cultured T-cells. The co-cultured T cells may then be screened to identify cancer reactive T-cells, i.e. T cells with at least one TCR specific to a cancer antigen.

### III. Enriching Activated T-Cell Populations

In various embodiments, to further enhance the probability of getting more tumor reactive T-iPSCs, activated T-cell populations may be enriched after co-culturing with a tumor antigen. The method may comprise screening the co-cultured T cells to identify cells expressing one or more T cell activation markers. When T cells are activated, T cells up-regulate the expression of one or more T cell activation markers. T cell activation occurs when the TCR and CD3 bind to antigen and major histocompatibility complex (MHC) and co-stimulatory molecules, as with CD28 binding to CD80 (B7-1) or CD86 (B7-2). Any one or more of a variety of T cell activation markers may be useful for separating the cancer reactive T cells from the other cells from the biological sample. Examples of T cell activation markers include, but are not limited to, any one or more of programmed cell death 1 (PD-1), lymphocyte-activation gene 3 (LAG-3), T cell immunoglobulin and mucin domain 3 (TIM-3), 4-1BB, OX40, CD107a, granzyme B, interferon (IFN)-γ, interleukin (IL)-2, tumor necrosis factor alpha (TNF-11), granulocyte/monocyte colony stimulating factor (GM-CSF), IL-4, IL-5, IL-9, IL-10, IL-17, IL-22, CD39, CD103 and TIGIT (T Cell Immunoreceptor With Ig And ITIM Domains). In aspects of the disclosure, the T cell activation markers are one or more cell surface markers of T cell activation. Such cell surface markers of T cell activation may include, for example, any one or more of PD-1, LAG-3, TIM-3, 4-1BB, OX40, CD107a, CD39, CD103 and TIGIT. In aspects, the co-cultured T-cells may be screened to identify cells expressing PD-1. Programmed cell death protein 1, also called PD-1 or CD279, is a protein on the surface of T cells that has a role in down regulating the immune response to autologous cells and promoting self-tolerance. Alternatively, or in addition, the co-cultured T cells may be screened to identify cells expressing 4-1BB. 4-1BB (or CD137) is a member of the tumor necrosis factor (TNF) receptor family. Screening for T cells expressing T cell activation markers, e.g. PD-1 and/or 4-1BB, may be carried out utilizing known methods.

Aspects of the disclosure may comprise isolating the cells expressing one or more T cell activation markers from the screened, co-cultured T-cells that do not express one or more T cell activation markers to obtain isolated cancer antigen-specific T cells. In this regard, the method may physically separate the cells that express one or more T cell activation markers from other cells from the co-culture that do not express the one or more T cell activation markers. The T cells that express one or more T cell activation markers may also express one or both of CD4 and CD8.

The isolating of the cells expressing one or more T cell activation markers may be carried out in any of a variety of different ways. In aspects of the disclosure, the isolating is carried out using flow cytometry. The flow cytometry may employ any suitable antibodies and stains. The antibody may be chosen such that it specifically recognizes and binds to the particular T cell activation marker under study. The antibody or antibodies may be conjugated to a bead (e.g., a magnetic bead) or to a fluorochrome. In an aspect, the separating may be carried out using fluorescence-activated cell sorting (FACS).

In aspects of the present disclosure, cancer-reactive T cells, for example, T cells expressing PD-1 and/or 4-1BB, are isolated to obtain isolated cancer reactive T cells. Suitable separation and isolation methods are known to those skilled in the art.

### IV. Reprogramming T-Cells to IPSCs

The method may further comprise culturing the isolated cancer reactive T cells under conditions sufficient produce an isolated population of iPSCs. iPSC technology allows the reprogramming of somatic cells, e.g. T cells, into an embryonic stem cell-like stage, which can be expanded indefinitely and retain the potential to differentiate into any type of somatic cell. Reprogramming of T cells, e.g. tumor infiltrating lymphocytes (TILs), may be desirable because the TCRs providing the antigen specificity of T cells are produced by genomic recombination and are thus inherited in the generated iPSC cell line. Accordingly, in aspects of the disclosure, cancer-reactive T cells reprogrammed into iPSCs yield a population of iPSCs comprising DNA encoding a cancer antigen-specific TCR.

In aspects, the isolated cancer reactive T cells are contacted with reprograming factors as disclosed herein. As used herein, the term “reprogramming factors” refers to any protein, polypeptide, amino acid, mRNA, DNA or small molecule capable of altering the differentiational state of a cell. Such reprogramming factors can include, but are not limited to the transcription factors, OCT4 (or OCT3/4), SOX3, KLF4 and c-Myc, discovered by Yamanaka and colleagues (see, e.g., Takahashi and Yamanaka, 2006, Cell, 136, 364-377) which are referred to herein as “OSKM” or the “Yamanaka factors.” Reprogramming factors refers to other factors that might alter the differentiation state of a cell, or that might enhance or alter the efficiency of cell reprogramming. Such factors are known in the art. Exemplary reprogramming factors are described in Feng et al. 2009, Cell Stem Cell Review, 4:301-313.

In various embodiments, not all of the four Yamanaka Factors are stringently necessary. In various embodiments, the isolated immune cells are contacted with OCT3/4 and SOX2. In various embodiments, the isolated immune cells are contacted with OCT3/4, SOX2 and c-myc. In various embodiments, the isolated cells are contacted with OCT3/4, SOX2 and KLF4. In one embodiment, the T cells are contacted with OCT3/4, SOX2 and KLF4, and either c-myc or SV40.

Additional reprogramming factors may be used in various embodiments to reprogram the isolated immune cells. Such factors include, but are not limited to, LIN28, NANOG, Esrrb, Pax5 shRNA, C/EBPa, p53 siRNA, UTF1, DNMT shRNA, Wnt3a, GLIS1, DLX4, CDH1, SV40 LT(T) and hTERT. In various embodiments, reprogramming factors may upregulate or downregulate certain miRNAs involved in reprogramming. In one embodiment, a reprogramming factor may upregulate or downregulate expression of one or more of the genes upstream or downstream of one or more of the Yamanaka Factors. In various embodiments, the one or more reprogramming factors may include, but are not limited to, histone methyltransferase inhibitors, L-type calcium channel agonists, DNA methyltransferase inhibitors, histone deacetylase inhibitors, MEK inhibitors, GSK3 inhibitors or TGF-B inhibitors. Any factor that modulates the upstream or downstream molecular pathway of the reprogramming transcription factors is contemplated for use in the reprogramming methods herein.

In various embodiments, the one or more reprogramming factors may be a small molecule.

The isolated cancer-reactive T cells can be cultured to produce an isolated population of iPSCs using any suitable technique. For example, the isolated cancer reactive T cells can receive stimulation from anti-CD3 and/or anti-CD28 antibodies. In aspects the isolated cancer reactive T cells may be transduced (e.g., with a vector) with sequences of the Yamanaka factors (i.e., Kruppel-like factor 4 (Klf4), Sry-related HMG-box gene 2 (Sox2), Octamer-binding transcription factor 3/4 (Oct3/4), and MYC protooncogene (c-Myc)) and SV40 (see, e.g., Vizcardo, et al., Cell Stem Cell, 12: 31-36 (2013)). In aspects, the cancer-reactive T cells may be cultured in the presence of interleukin-2 (IL-2), interleukin-7 (IL-7), interleukin-15 (IL-15), interleukin-12 (IL-12), or a combination of two or more of the foregoing. In other aspects, the isolated population of iPSCs can be generated by RNA expression, protein delivery, chemical induction of reprogramming genes, activation by upstream or downstream targeting of gene pathways essential for reprogramming (e.g. Sox2, KLf4, Oct4, Nanog, etc.), or any combination of these methods.

In certain aspects, isolated cancer-reactive T cells are contacted with reprogramming factors under conditions sufficient to reprogram the cells into a pluripotent stem cell stage, thereby yielding an isolated population of iPSCs comprising nucleotide sequences encoding a cancer antigen-specific TCR or TCRs. In aspects, the reprogramming factors comprise one or more of Kruppel-like factor 4 (Klf4), Sry-related HMG-box gene 2 (Sox2), Octamer-binding transcription factor 3/4 (Oct3/4), MYC protooncogene (c-Myc)) and Large T Antigen (SV40).

In aspects of the present disclosure, the reprogramming will begin within a short time after the conclusion of the co-culturing of the isolated T-cells with the isolated cancer cells. For example, in some aspects, the reprogramming step will begin within 24 hours of the conclusion of the co-culturing. In other aspects, the reprogramming begin within about 20 hours of the conclusion of the co-culturing. In other aspects, the reprogramming will begin within about 16 hours of the conclusion of the co-culturing. In other aspects, the reprogramming step will begin within about 12 hours of the conclusion of the co-culturing. In other aspects, the reprogramming step will begin within about 8 hours of the conclusion of the co-culturing. In other aspects, the reprogramming will begin within about 6 hours of the conclusion of the co-culturing. In other aspects, the reprogramming step will begin within about 2 hours of the conclusion of the co-culturing. In certain aspects the screening, isolating and reprogramming of the cancer-reactive T cells are carried out sequentially immediately after the completion of the co-culture step.

In various embodiments of the present disclosure, T-cells are first co-cultured with a tumor antigen, then those co-cultured T-cells expressing activation markers (i.e., activated T-cells) are isolated, and only the activated T-cells are reprogrammed. In various embodiments, the activated T-cells are enriched immediately following the conclusion of co-culturing. For example, in some aspects, the enrichment step will begin within 24 hours of the conclusion of the co-culturing. In other aspects, the enrichment step will begin within about 20 hours of the conclusion of the co-culturing. In other aspects, the enrichment step will begin within about 16 hours of the conclusion of the co-culturing. In other aspects, the enrichment step will begin within about 12 hours of the conclusion of the co-culturing. In other aspects, the reprogramming step will begin within about 8 hours of the conclusion of the co-culturing. In other aspects, the reprogramming will begin within about 6 hours of the conclusion of the co-culturing. In other aspects, the reprogramming step will begin within about 2 hours of the conclusion of the co-culturing. In certain aspects the screening, isolating and reprogramming of the cancer-reactive T cells are carried out sequentially immediately after the completion of the co-culture step.

In some aspects, the reprogramming step will begin within 24 hours of the conclusion of the enrichment step. In other aspects, the reprogramming begin within about 20 hours of the conclusion of the enrichment step. In other aspects, the reprogramming will begin within about 16 hours of the conclusion of the enrichment step. In other aspects, the reprogramming step will begin within about 12 hours of the conclusion of the enrichment step. In other aspects, the reprogramming step will begin within about 8 hours of the conclusion of the co-culturing. In other aspects, the reprogramming will begin within about 6 hours of the conclusion of the enrichment step. In other aspects, the reprogramming step will begin within about 2 hours of the conclusion of the enrichment step. In certain aspects the screening, isolating and reprogramming of the cancer-reactive T cells are carried out sequentially immediately after the completion of the enrichment step.

### VI. Identification of Novel TCR Sequences

Aspects of the disclosure include methods for determining a nucleotide sequence encoding an antigen-specific TCR. Such methods may comprise (a) producing an isolated population of iPSCs according to any of the methods described herein with respect to other aspects of the invention; and (b) determining the nucleotide sequence encoding the cancer antigen-specific TCR by performing RNA or DNA sequencing of nucleic acids comprised in the isolated population of iPSCs. Such methods may also comprise differentiating the iPSCs into T lineage cells expressing the cancer antigen-specific TCR to obtain differentiated T lineage cells and determining the nucleotide sequence encoding the cancer antigen-specific TCR by performing RNA or DNA sequencing of nucleic acid comprised in the isolated population of T lineage cells. The methods disclosed herein allow for the identification of tumor antigen specific TCRs of low frequency that were unable to be detected by prior art methods.

In various embodiments, the disclosure provides a method of identifying a tumor antigen specific TCR, comprising isolating T cells from a first sample from the subject, wherein the subject has cancer, contacting the isolated T-cells with one or more tumor antigens to produce co-cultured T-cells; isolating from the co-cultured T-cells those expressing one or more T cell activation markers, contacting the isolating T cells with one or more reprogramming factors under conditions sufficient to reprogram the cells into T-iPSCs; and determining the DNA sequence encoding the TCR alpha and TCR beta chain from an iPSC colony.

Examples of sequencing techniques that may be useful in the disclosed methods include Next Generation Sequencing (NGS) (also referred to as “massively parallel sequencing technology” or “deep sequencing”) and Third Generation Sequencing. NGS refers to non-Sanger-based high-throughput DNA sequencing technologies. With NGS, millions or billions of DNA strands may be sequenced in parallel, yielding substantially more throughput and minimizing the need for the fragment-cloning methods that are often used in Sanger sequencing of genomes. In NGS, nucleic acid templates may be randomly read in parallel along the entire genome by breaking the entire genome into small pieces. NGS may, advantageously, provide nucleic acid sequence information in very short time periods, e.g., within about 1 to about 2 weeks, within about 1 to about 7 days, or within less than about 24 hours. Multiple NGS platforms which are commercially available or which are described in the literature can be used in the context of the disclosed methods, e.g., those described in Zhang et al., J. Genet. Genomics, 38(3): 95-109 (2011) and Voelkerding et al., Clinical Chemistry, 55: 641-658 (2009).

Non-limiting examples of NGS technologies and platforms include sequencing-by-synthesis (also known as “pyrosequencing”) (as implemented, e.g., using the GS-FLX 454 Genome Sequencer, 454 Life Sciences (Branford, Conn.), SOLEXA Genome Analyzer (Illumina Inc., San Diego, Calif.), the HISEQ 2000 Genome Analyzer (Illumina), the NEXTSEQ system (Illumina), the MISEQ system (Illumina) or as described in, e.g., Ronaghi et al., Science, 281(5375): 363-365 (1998)), sequencing-by-ligation (as implemented, e.g., using the SOLID platform (Life Technologies Corporation, Carlsbad, Calif.) or the POLONATOR G.007 platform (Dover Systems, Salem, N.H.)), single-molecule sequencing (as implemented, e.g., using the PACBIO RS system (Pacific Biosciences (Menlo Park, Calif.) or the HELISCOPE platform (Helicos Biosciences (Cambridge, Mass.)), nano-technology for single-molecule sequencing (as implemented, e.g., using the GRIDON platform of Oxford Nanopore Technologies (Oxford, UK), the hybridization-assisted nano-pore sequencing (HANS) platforms developed by Nabsys (Providence, R.I.), and the ligase-based DNA sequencing platform with DNA nanoball (DNB) technology referred to as probe-anchor ligation (cPAL)), electron microscopy-based technology for single-molecule sequencing, and ion semiconductor sequencing.

In various embodiments, the identified TCR sequences are further screened to determine antigen reactivity. In various embodiments, peripheral blood mononuclear cells (PBMCs) are transduced with an expression vector comprising the sequence of the TCR alpha and TCR beta chains identified; and contacted with one or more tumor antigens associated with the cancer. Next, reactivity of the transduced PBMCs to the one or more tumor antigens is measured to confirm that the TCR is tumor antigen-specific. Techniques for measuring reactivity are described in the art.

In various embodiments, the TCR is rare tumor antigen specific TCR. As used herein, a “rare tumor antigen specific TCR” refers to a tumor antigen that has a relatively low frequency in a heterologous population of antigen-specific T-cells. In various embodiments, the heterologous population of antigen-specific T cells are derived from a tumor. In various embodiments, the heterologous population of antigen-specific T cells are derived from a tumor infiltrating lymphocyte (TIL). In various embodiments, a rare tumor antigen specific TCR represents less than 9%, 8%, 7%, 6%, 5%, 4%, 3%, 2%, 1%, or 0.1% of the heterologous population of antigen-specific T-cells (i.e. the bulk).

In various embodiments, the novel TCR sequences identified herein may be used to produce genetically engineered cells for adoptive cell therapy for the treatment of cancer. In certain embodiments, PBMC are transduced with an expression vector (such as a viral vector) encoding a tumor antigen-specific TCR identified herein. In another embodiment, any biological sample containing T, NK or NKT cells, such as from a tumor biopsy or TILs, are suitable for being transduced to express the TCRs disclosed herein. In one embodiment, isolated T cells from a subject with cancer are transduced with an expression vector encoding a TCR specific to a tumor antigen as disclosed herein. In various embodiments, the engineered T cells have been genetically modified to remove the endogenous TCR. Methodology for engineering TCR sequences into T cells are disclosed in the art. See, e.g., Mol. Ther. 28(1) 8 Jan. 2020, 64-74; Blood 2018; 131(3): 311-322; Life Sci Alliance, 2019 April; 2(2): e201900367; Nature. 2017 Mar. 2; 543(7643): 113-117. Certain methods for making the constructs and engineered T cells of the disclosure are described in PCT application PCT/US2015/14520. Additional methods of making the constructs and cells can be found in U.S. provisional patent application No. 62/244,036.

For cloning of polynucleotides, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marc-escans, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different recombinant TCR as disclosed herein. The resulting transduced immune effector cells form a mixed population of engineered cells, with a proportion of the engineered cells expressing more than one different recombinant TCR.

In various embodiments, the engineered T cells are autologous T cells. In various embodiments the engineered T cells are allogeneic. In various embodiments, the novel TCR sequence are used to generate an allogenic cell line for the treatment of cancer or a tumor. In various embodiments, the present disclosure relates to a method of producing a medicament for the treatment of cancer in a subject having cancer, the method comprising, producing an isolated population of iPSCs using the methods described herein, identifying a novel TCR sequence, expressing the Va and Vb sequences of said TCR sequence in a population of T cells and formulating the engineered T cells into a medicament for the treatment or prevention of cancer in a subject.

In various embodiments, the endogenous TRAC locus has been replaced with a nucleic acid sequence encoding the TCR comprising the CDR3 Va region of any one of SEQ ID Nos 1-4 and a CDR3 Vb region of any one of SEQ ID NOs. 5-8 and the TCRB locus has been knocked out. In various embodiments, the endogenous TRAC locus has been replaced with a nucleic acid sequence encoding the TCR comprising the CDR3 Va region of SEQ ID No. 1 and a CDR3 Vb region of any one of SEQ ID No. 5. In various embodiments, the endogenous TRAC locus has been replaced with a nucleic acid sequence encoding the TCR comprising the CDR3 Va region of SEQ ID No. 2 and a CDR3 Vb region of any one of SEQ ID NO. 6. In various embodiments, the endogenous TRAC locus has been replaced with a nucleic acid sequence encoding the TCR comprising the CDR3 Va region of SEQ ID No. 3 and a CDR3 Vb region of any one of SEQ ID No 7. In various embodiments, the endogenous TRAC locus has been replaced with a nucleic acid sequence encoding the TCR comprising the CDR3 Va region of SEQ ID No 4 and a CDR3 Vb region of any one of SEQ ID No 8. In certain embodiments, the nucleic acid sequences encoding the TCR also comprise a nucleic acid encoding an appropriate constant region. Constant regions suitable for use herein are known in the art (see for example, UniProt P01848 (TRAC), P01850 (TRBC1), UniProtKB—A0A0G2JMB4 (TRBC2).

In various embodiments, the disclosure provides for a recombinant TCR or a chimeric TCR comprising a V alpha chain of SEQ ID NO:9 and a V beta chain of SEQ ID NO:13. In various embodiments, the disclosure provides for a recombinant TCR or a chimeric TCR comprising a V alpha chain of SEQ ID NO:10 and a V beta chain of SEQ ID NO:14. In various embodiments, the disclosure provides for a recombinant TCR or a chimeric TCR comprising a V alpha chain of SEQ ID NO:11 and a V beta chain of SEQ ID NO:15. In various embodiments, the disclosure provides for a recombinant TCR or a chimeric TCR comprising a V alpha chain of SEQ ID NO:12 and a V beta chain of SEQ ID NO:16. In various embodiments, the disclosure provides for an isolated cell expressing the recombinant or chimeric TCRs comprising any one of SEQ ID NOs: 9-12.

V. Methods of Establishing Tumor Specific iPSC Lines

In various embodiments, the method provides for making tumor specific iPSC lines. In various embodiments, the method preferentially reprograms tumor reactive T cells from heterologous populations of T cells. In various embodiments, the tumor antigen specific T-iPSC lines may be advantageous in developing novel cell therapies for the treatment of various cancers. In certain embodiments, T-iPSC colonies may be maintained iPSC lines in appropriate commercially available media for maintaining stem cells using established protocols (see e.g., Curr Protoc Stem Cell Biol. 2020 September; 54(1):e117. doi: 10.1002/cpsc.117; Methods Mol Biol. 2021 Apr. 10. doi: 10.1007/7651_2021_358). Such T-iPSC lines are generally monoclonal with respect to their rearranged TCR genes (from the tumor antigen-specific T cell from which they were reprogrammed). In certain embodiments, multiple such T-iPSC lines may be pooled to generate polyclonal T-iPSC lines.

Aspects of the disclosure include methods for treating cancer in a subject using the disclosed isolated populations of iPSCs comprising one or more nucleotide sequences encoding a cancer antigen-specific TCR. Such methods may comprise (a) producing an isolated population of iPSCs as disclosed herein with respect to other aspects of the invention; (b) differentiating the iPSCs into T lineage cells, to obtain differentiated T lineage cells; and (c) administering the differentiated T lineage cells to the subject in an amount effective to treat or prevent cancer in the subject. In aspects, the differentiated T lineage cells may be naïve T cells.

The differentiation of T-iPSCs into T lineage cells can be achieved using methods disclosed in the art. See, e.g., Restifo, N. P., Dudley, M. E., and Rosenberg, S. A. (2012). Nat. Rev. Immunol., 12, 269-281. In various embodiments, the method may further comprise culturing the T-iPSCs to produce CD4−CD8− (double negative) T cells. Double negative T cells do not express the CD4 or CD8 co-receptor. Double negative T cells are differentiated from common lymphoid progenitor (CLP) cells and engraft in the thymus. The T-iPSCs can be cultured to produce CD4−CD8− (double negative) T cells using any suitable technique.

The populations of cells produced according to the disclosure can be used in methods of treating or preventing cancer in a subject. In this regard, aspects of the disclosure include a method of treating or preventing cancer in a subject, comprising (i) administering to the subject the cells produced according to any of the methods described herein or (ii) administering to the subject any of the isolated populations of cells or pharmaceutical compositions described herein; in an amount effective to treat or prevent cancer in the subject.

### VII. Methods of Treatment and Pharmaceutical Compositions

In aspects of the disclosure, the method of treating or preventing cancer may comprise administering the cells or pharmaceutical composition to the subject in an amount effective to reduce metastases in the subject. For example, the disclosed methods may reduce metastatic nodules in the subject.

The cells administered to the subject can be allogeneic or autologous to the subject. In “autologous” administration methods, cells are removed from a subject, stored (and optionally modified), and returned back to the same subject. In “allogeneic” administration methods, a subject receives cells from a genetically similar, but not identical, donor. In aspects, the T cells are autologous to the subject. Autologous cells may, advantageously, reduce or avoid the undesirable immune response that may target an allogeneic cell such as, for example, graft-versus-host disease.

For purposes of the disclosure, the dose, e.g., number of cells administered should be sufficient to effect, e.g., a therapeutic or prophylactic response, in the subject over a reasonable time frame. For example, the number of cells administered should be sufficient to bind to an antigen of interest or treat or prevent cancer in a period of from about 2 hours or longer, e.g., 12 to 24 or more hours, from the time of administration. In certain aspects, the time period could be even longer. The number of cells administered will be determined by, e.g., the efficacy of the particular population of cells to be administered and the condition of the animal (e.g., human), as well as the body weight of the animal (e.g., human) to be treated.

The number of cells administered also will be determined by the existence, nature and extent of any adverse side effects that might accompany the administration of a particular population of cells. Typically, the attending physician will decide the number of cells with which to treat each individual subject, taking into consideration a variety of factors, such as age, body weight, general health, diet, sex, route of administration, and the severity of the condition being treated. By way of example and without limitation, the number of cells, e.g., differentiated T lineage cells or cells engineered to express a tumor antigen specific TCR identified as described herein, to be administered can be about 10×10⁶ to about 10×10¹¹ cells per infusion, about 10×10⁹ cells to about 10×10¹¹ cells per infusion, or 10×10⁷ to about 10×10⁹ cells per infusion.

One or more additional therapeutic agents can be co-administered to the subject. Use of “co-administering” herein means administering one or more additional therapeutic agents and the isolated population of cells sufficiently close in time such that the isolated population of cells can enhance the effect of one or more additional therapeutic agents, or vice versa. In this regard, the isolated population of cells can be administered first and the one or more additional therapeutic agents can be administered second, or vice versa. Alternatively, the isolated population of cells and the one or more additional therapeutic agents can be administered simultaneously. Additional therapeutic agents that may enhance the function of the isolated population of cells may include, for example, one or more cytokines or one or more antibodies (e.g., antibodies that inhibit PD-1 function). An exemplary therapeutic agent that can be co-administered with the isolated population of cells is IL-2. Without being bound to a particular theory or mechanism, it is believed that IL-2 may enhance the therapeutic effect of the isolated population of cells, e.g., differentiated T lineage cells.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the disclosed methods can provide any amount of any level of treatment or prevention of cancer in a subject. Furthermore, the treatment or prevention provided by the disclosed methods can include treatment or prevention of one or more conditions or symptoms of the cancer being treated or prevented. Also, for purposes herein, “prevention” can encompass delaying the onset or recurrence of the cancer, or a symptom or condition thereof.

In aspects of the disclosure, the method of treating or preventing cancer may comprise administering the cells or pharmaceutical composition to the subject in an amount effective to reduce metastases in the subject. For example, the disclosed methods may reduce metastatic nodules in the subject

Aspects of the present disclosure include methods for producing a medicament for the treatment or prevention of cancer in a subject having cancer using the disclosed isolated populations of cells. Such methods may comprise the steps (a) producing an isolated population of iPSCs as disclosed herein (b) differentiating the iPSCs into T lineage cells, to obtain differentiated T lineage cells; and (c) formulating the differentiated T lineage cells into a medicament for the treatment or prevention of cancer in the subject. In aspects, the differentiated T lineage cells may be naïve T cells. Such methods may comprise the steps (a) producing an isolated population of T-iPSCs as disclosed herein (b) determining the sequence of the antigen-specific TCR; (c) transducing a population of T cells with a vector expressing the TCR; and (d) formulating the TCR engineered T cells into a medicament for the treatment or prevention of cancer in the subject.

The isolated populations of cells produced according to the disclosed methods (e.g. the isolated populations of iPSCs, and/or the differentiated T lineage cells, and/or the TCR engineered T cells) may be included in a composition, such as a pharmaceutical composition. In this regard, aspects of the disclosure include a pharmaceutical composition comprising the isolated or purified population of cells described herein and a carrier. In aspects, the composition may comprise iPSCs comprising one or more nucleotide sequences encoding one or more cancer antigen-specific TCRs. In aspects, the composition may comprise differentiated T lineage cells obtained by differentiating such iPSCs. Aspects of the present disclosure may include compositions for use in the treatment or prevention of cancer. Such compositions may comprise, for example, differentiated T lineage cells prepared by differentiating iPSCs as disclosed herein.

In aspects, compositions according to the present disclosure may comprise a carrier. The carrier may be a pharmaceutically acceptable carrier. With respect to pharmaceutical compositions, the carrier can be any of those conventionally used for the administration of cells. Such pharmaceutically acceptable carriers are well known to those skilled in the art and are readily available to the public. In aspects, the pharmaceutically acceptable carrier has no detrimental side effects or toxicity under the conditions of use.

The choice of carrier will be determined in part by the particular method used to administer the population of cells. Accordingly, there are a variety of suitable formulations of the pharmaceutical composition according to the present disclosure. Suitable formulations may include any of those for parenteral, subcutaneous, intravenous, intramuscular, intraarterial, intrathecal, intratumoral, or intraperitoneal administration. More than one route can be used to administer the population of cells, and in certain instances, a particular route can provide a more immediate and more effective response than another route.

A population of cells may be administered by injection, e.g., intravenously. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL electrolyte solution (Abbott, Chicago, Ill.), PLASMA-LYTE A (Baxter, Deerfield, Ill.), about 5% dextrose in water, or Ringer's lactate. In aspects, the pharmaceutically acceptable carrier is supplemented with human serum albumen.

The terms “nucleic acid” and “polynucleotide,” as used herein, refer to a polymeric form of nucleotides of any length, either ribonucleotides (RNA) or deoxyribonucleotides (DNA). These terms refer to the primary structure of the molecule, and thus include double- and single-stranded DNA, double- and single-stranded RNA, and double-stranded DNA-RNA hybrids. The terms include, as equivalents, analogs of either RNA or DNA made from nucleotide analogs and modified polynucleotides such as, though not limited to, methylated and/or capped polynucleotides. In aspects of the disclosure, the nucleic acid is complementary DNA (cDNA).

The term “nucleotide” as used herein refers to a monomeric subunit of a polynucleotide that consists of a heterocyclic base, a sugar, and one or more phosphate groups. The naturally occurring bases (guanine (G), adenine (A), cytosine (C), thymine (T), and uracil (U)) are typically derivatives of purine or pyrimidine, though the disclosure includes the use of naturally and non-naturally occurring base analogs. The naturally occurring sugar is the pentose (five-carbon sugar) deoxyribose (which forms DNA) or ribose (which forms RNA). Nucleic acids are typically linked via phosphate bonds to form nucleic acids or polynucleotides, though many other linkages are known in the art (e.g., phosphorothioates, boranophosphates, and the like). Methods of preparing polynucleotides are within the ordinary skill in the art (Green and Sambrook, Molecular Cloning: A Laboratory Manual, (4th Ed.) Cold Spring Harbor Laboratory Press, New York (2012)).

In aspects, the isolated population of iPSCs comprises iPSCs that comprise one or more nucleotide sequences encoding one or more antigen-specific TCRs.

In aspects of the disclosure, the TCRs of the differentiated T lineage cells have antigenic specificity for a cancer (i.e., a cancer antigen). In further aspects of the disclosure, the TCRs of the differentiated T lineage cells specifically bind to the one or more cancer antigens of cancer cells. In aspects, the cancer antigen is a tumor antigen.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, e.g., both breast and prostate cancer cells may express the cancer antigen. Cancer antigens are known in the art and include, for instance, CXorf61, mesothelin, CD19, CD22, CD276 (B7H3), gp100, MART-1, Epidermal Growth Factor Receptor Variant III (EGFRVIII), TRP-1, TRP-2, tyrosinase, NY-ESO-1 (also known as CAG-3), MAGE-1, MAGE-3, etc.

### Aspects of the Disclosure

Aspects of the present subject matter described herein may be beneficial alone or in combination, with one or more other aspects. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of producing an isolated population of tumor antigen specific T-cell induced pluripotent stem cells (T-iPSCs), the method comprising:
(a) isolating T cells from a first sample from a subject, wherein said subject has cancer;
(b) contacting the isolated T-cells of (a) with one or more tumor antigens to produce co-cultured T-cells;
(c) isolating from the co-cultured T-cells, T-cells expressing one or more T cell activation markers; and
(d) contacting the isolated T-cells of (c) with one or more reprogramming factors under conditions sufficient to reprogram the cells into T-iPSCs.
2. The method of aspect 1, wherein the first sample is a tumor sample or PBMC.
3. The method of aspect 1, wherein the isolated T cells of step (a) are tumor infiltrating lymphocytes.
4. The method according to aspect 1, wherein the co-culturing proceeds for about 3-48 hours.
5. The method according to aspect 1, wherein the co-culturing proceeds for about 8-48 hours.
6. The method according to aspect 1, wherein the co-culturing proceeds for about 8-24 hours.
7. The method according to aspect 1, wherein the co-culturing proceeds for about 12-20 hours.
8. The method according to aspect 1, wherein the co-culturing proceeds for about 16 hours.
9. The method according to any one of aspects 1-8, wherein the tumor antigen is derived from cancer cells.
10. The method according to aspect 9, wherein the cancer cells are isolated from said subject.
11. The method according to any one of aspects 1-8, wherein the isolated T cells are co-cultured with cancer cells expressing said tumor antigen.
12. The method according to any one of aspects 1-8, wherein the isolated T cells are co-cultured with autologous tumor cells.
13. The method according to any one of aspects 1-8, wherein the isolated T cells are co-cultured with irradiated tumor cells.
14. The method according to any one of aspects 1-8, wherein the isolated T cells are co-cultured with a tumor cell line.
15. The method according to any one of aspects 1-8, wherein the isolated T cells are co-cultured with peptide pools, wherein the peptide pools are derived from the tumor antigen.
16. The method of any one of aspects 1-11, wherein the one or more T cell activation marker(s) includes PD-1 or 4-1BB.
17. The method according to any one of aspects 1-12, wherein the reprogramming factors comprise one or more of: Kruppel-like factor 4 (Klf4), Sry-related HMG-box gene 2 (Sox2), Octamer-binding transcription factor 3/4 (Oct3/4), MYC protooncogene (c-Myc)) and Large T Antigen (SV40).
18. The method according to any one of aspects 1-14, wherein the subject is a mammal.
19. The method according to aspect 15, wherein the mammal is human.
20. The method according to any one of aspects 1-16, wherein the cancer comprises a solid tumor.
21. The method according to any one of aspects 1-17, further comprising differentiating iPSCs in the isolated population of iPSCs into T lineage cells to obtain differentiated T lineage cells.
22. A method of producing a medicament for the treatment or prevention of cancer in a subject having cancer, the method comprising:
(a) producing an isolated population of iPSCs according to the method of any one of aspects 1-17;
(b) differentiating the iPSCs into T lineage cells, to obtain differentiated T lineage cells; and
(c) formulating the differentiated T lineage cells into a medicament for the treatment or prevention of cancer in the subject.
23. A method of identifying a cancer antigen-specific TCR, the method comprising:
(a) producing an isolated population of iPSCs according to the method of any one of aspects 1-17; and
(b) determining the nucleotide sequence encoding the cancer antigen-specific TCR by performing RNA or DNA sequencing of nucleic acids comprised in the isolated population of iPSCs.
24. The method of aspect 23, wherein the nucleotide sequence encoding the cancer-antigen specific TCR is determined by performing DNA sequencing.
25. The method of aspect 23, wherein the nucleotide sequence encoding the cancer-antigen specific TCR is determined by ImmunoSEQ.
26. A composition comprising the iPSCs produced according to the method of any one of aspects 1-17 and a pharmaceutically acceptable carrier.
27. A composition comprising the differentiated T lineage cells produced according to the method of aspect 19 and a pharmaceutically acceptable carrier.
28. The isolated population of iPSCs produced according to the method of any one of aspects 1-17, or the composition of aspect 26, for use in the treatment or prevention of cancer in the subject having cancer.
29. The isolated population of iPSCs or the composition for the use of aspect 24, wherein the subject is a mammal.
30. The isolated population of iPSCs or the composition for the use of aspect 25, wherein the mammal is a human.
31. The T lineage cells produced according to the method of aspect 19, or the composition of aspect 23, for use in the treatment or prevention of cancer in a subject having cancer.
32. The T lineage cells or the composition for the use of aspect 27, wherein the subject is a mammal.
33. The T lineage cells or the composition for the use of aspect 28, wherein the mammal is a human.
34. A method of identifying a tumor antigen specific TCR, comprising:
(a) isolating T cells from a first sample from the subject, wherein said subject has cancer;
(b) contacting the isolated T-cells of (a) with one or more tumor antigens to produce co-cultured T-cells;
(c) isolating from the co-cultured T-cells of (b) T-cells expressing one or more T cell activation markers;
(d) contacting the isolated T-cells of (c) with one or more reprogramming factors under conditions sufficient to reprogram the cells into T-iPSCs; and
(e) determining the DNA sequence encoding the TCR alpha and TCR beta chain from an iPSC colony.
35. The method of aspect 34, further comprising the steps of
(f) transducing a peripheral blood mononuclear cells (PBMC) with an expression vector comprising the sequence of the TCR alpha and TCR beta chains of (e); and
(g) contacting the transduced PBMCs with one or more tumor antigens associated with the cancer; and
(h) measuring reactivity of the transduced PBMC to the one or more tumor antigens;
wherein reactivity confirms that the TCR is tumor antigen-specific.
36. The method of aspect 35, further wherein the reactivity is measured by upregulation of one or more T cell activation markers.
37. The method of aspect 35, further wherein the reactivity is measured by production of one or more cytokines.
38. The method of aspect 35, further wherein the reactivity is measured by the ability of the transduced PBMC to kill a tumor cell expressing the tumor antigen.
39. The method of aspect 34-38, wherein the first sample is a tumor sample.
40. The method of aspect 34-38, wherein the isolated T cells of step (a) are tumor infiltrating lymphocytes.
41. The method according to aspect 34-38, wherein the contacting proceeds for about 3-48 hours.
42. The method according to aspect 34-38, wherein the contacting proceeds for about 8-48 hours.
43. The method according to aspect 34-38, wherein the contacting proceeds for about 8-24 hours.
44. The method according to aspect 34-38, wherein the contacting proceeds for about 12-20 hours.
45. The method according to aspect 34-38, wherein the contacting proceeds for about 16 hours.
46, The method according to any one of aspects 34-45, wherein the tumor antigen is derived from cancer cells.
47. The method according to aspects 34-45, wherein the cancer cells are isolated from said subject.
48. The method according to any one of aspects 34-45, wherein the isolated T cells are co-cultured with cancer cells expressing said tumor antigen.
49. The method of any one of aspects 34-45, wherein the one or more T cell activation marker(s) includes PD-1 or 4-1BB.
50. The method according to any one of aspects 34-45, wherein the reprogramming factors comprise one or more of: Kruppel-like factor 4 (Klf4), Sry-related HMG-box gene 2 (Sox2), Octamer-binding transcription factor 3/4 (Oct3/4), MYC protooncogene (c-Myc)) and Large T Antigen (SV40).
51. The method according to any one of aspects 34-45, wherein the tumor antigen is derived from cancer cells.
52. The method according to aspect 51, wherein the cancer cells are isolated from said subject.
53. The method according to any one of aspects 34-45, wherein the isolated T cells are co-cultured with cancer cells expressing said tumor antigen,
54. The method according to any one of aspects 34-45, wherein the isolated T cells are co-cultured with autologous tumor cells
55. The method according to any one of aspects 34-45, wherein the isolated T cells are co-cultured with irradiated tumor cells.
56. The method according to any one of aspects 34-45, wherein the isolated T cells are co-cultured with a tumor cell line.
57. The method according to any one of aspects 34-45, wherein the isolated T cells are co-cultured with peptide pools, wherein the peptide is the tumor antigen.
58. The method of aspect 34 wherein the tumor antigen specific TCR represents less than 9%, 8%, 7%, 6%, 5%, 4%, 3%, 2%, 1%, or 0.1% of the isolated T-cells from the first sample from the subject.
59. The method of aspect 58, wherein the first sample is a tumor sample or PBMC.
60. The method of aspect 58, wherein the isolated T cells of step (a) are tumor infiltrating lymphocytes.
61. A method of generating a polyclonal population of tumor antigen specific iPSC derived T cells, comprising:
(a) isolating T cells from a first sample from the subject, wherein said subject has cancer;
(b) contacting the isolated T-cells of (a) with one or more tumor antigens to produce co-cultured T-cells;
(c) isolating from the co-cultured T-cells of (b) T-cells expressing one or more T cell activation markers;
(d) contacting the isolated T-cells of (c) with one or more reprogramming factors under conditions sufficient to reprogram the cells into T-iPSCs; and
(e) differentiating the iPSCs into T lineage cells, to obtain differentiated T lineage cells.
62. The method of aspect 61, wherein the first sample is a tumor sample or PBMC.
63. The method of aspect 61, wherein the isolated T cells of step (a) are tumor infiltrating lymphocytes.
64. The method according to aspect 61, wherein the co-culturing proceeds for about 3-48 hours.
65. The method according to aspect 61, wherein the co-culturing proceeds for about 8-48 hours.
66. The method according to aspect 61, wherein the co-culturing proceeds for about 8-24 hours.
67. The method according to aspect 61, wherein the co-culturing proceeds for about 12-20 hours.
68. The method according to aspect 61, wherein the co-culturing proceeds for about 16 hours.
69. The method according to any one of aspects 61-68, wherein the tumor antigen is derived from cancer cells.
70. The method according to aspect 69, wherein the cancer cells are isolated from said subject.
71. The method according to any one of aspects 61-70, wherein the isolated T cells are co-cultured with cancer cells expressing said tumor antigen.
72. The method according to any one of aspects 61-70, wherein the isolated T cells are co-cultured with autologous tumor cells.
73. The method according to any one of aspects 61-70, wherein the isolated T cells are co-cultured with irradiated tumor cells.
74. The method according to any one of aspects 61-70, wherein the isolated T cells are co-cultured with a tumor cell line.
75. The method according to any one of aspects 61-70, wherein the isolated T cells are co-cultured with peptide pools, wherein the peptide pools are derived from the tumor antigen.
76. The method of any one of aspects 61-75, wherein the one or more T cell activation marker(s) includes PD-1 or 4-1BB.
77. The method according to any one of aspects 61-76, wherein the reprogramming factors comprise one or more of: Kruppel-like factor 4 (Klf4), Sry-related HMG-box gene 2 (Sox2), Octamer-binding transcription factor 3/4 (Oct3/4), MYC protooncogene (c-Myc)) and Large T Antigen (SV40).
78. The method according to any one of aspects 61-77, wherein the subject is a mammal.
79. The method according to aspect 78, wherein the mammal is human.
80. The method according to any one of aspects 61-79, wherein the cancer comprises a solid tumor.
81. A nucleic acid sequence encoding an amino acid sequence selected from SEQ ID NOs 1-16.
82. A recombinant TCR comprising:
(a) a CDR3 Va region of any one of SEQ ID Nos 1-4 and a CDR3 Vb region of any one of SEQ ID NOs. 5-8;
(b) A V alpha chain of SEQ ID NO:9 and a V beta chain of SEQ ID NO:13;
(c) A V alpha chain of SEQ ID NO:10 and a V beta chain of SEQ ID NO:14;
(d) V alpha chain of SEQ ID NO:11 and a V beta chain of SEQ ID NO:15; or
(e) V alpha chain of SEQ ID NO:12 and a V beta chain of SEQ ID NO:16.
83. A chimeric TCR comprising:
(a) a CDR3 Va region of any one of SEQ ID Nos 1-4 and a CDR3 Vb region of any one of SEQ ID NOs. 5-8;
(b) A V alpha chain of SEQ ID NO:9 and a V beta chain of SEQ ID NO:13;
(c) A V alpha chain of SEQ ID NO:10 and a V beta chain of SEQ ID NO:14;
(d) V alpha chain of SEQ ID NO:11 and a V beta chain of SEQ ID NO:15; or
(e) V alpha chain of SEQ ID NO:12 and a V beta chain of SEQ ID NO:16.
84. An isolated cell expressing a recombinant TCR of aspect 82 or a chimeric TCR of aspect 83.
85. The isolated cell of aspect 84 wherein the isolated cell is a human T cell.
86. The isolated cell of aspect 85 wherein the human T cell has been genetically modified to remove the endogenous TCR.
87. The isolated cell of aspect 85 wherein the endogenous TRAC locus has been replaced with a nucleic acid sequence encoding the TCR comprising the CDR3 Va region of any one of SEQ ID Nos 1-4 and a CDR3 Vb region of any one of SEQ ID NOs. 5-8 and the TCRB locus has been knocked out.

### 1. EXAMPLES

The following examples should not be construed as in any way limiting the scope of the disclosure or claims.

The following materials and methods were employed for the experiments described in Examples 1-3.

### Selecting Subjects, TIL and Autologous Tumor Lines

Frozen tumor infiltrating lymphocyte (TIL) samples were obtained from the NCI Surgery Branch TIL lab repository. TILs were generated as previously described. (See Tran et al., Nat Immunol. 2017; 18:255-62; see also Gross et al., J Clin Invest. 2014; 124:2246-59; see also Pasetto et al., Cancer Immunol Res. 2016; 4:734-43.) Briefly, surgically resected tumors were cut into approximately 1-2 mm fragments and cultured in complete media (CM) containing high dose IL-2 (6000 IU/ml). TIL fragment cultures were frozen after short time culture (day 13-16). The TILs of one subject designated 4069 were further screened for neoantigen reactivity by tandem minigene screening (see Tran, supra) and reactive TILs were expanded in the presence of irradiated feeder cells, 50 ng OKT-3 and 3000 IU IL-2 in 50-50 media (RPMI-AIM-V with 5% human AB serum with pen strep and L-glutamine) to reach approximately 100-150 billion cells for infusion and frozen.

Matched melanoma cell lines carrying a mutation specific antigen were established from fragment culture and were cultured in RPMI 1640 medium supplemented with 10% FBS (Gibco) at 37° C. and 5% CO₂. Melanoma cell lines were mycoplasma negative and were authenticated based on the identification of patient-specific somatic mutations and HLA molecules. All patients had undergone prior therapies including surgery, chemotherapy and immunotherapy.

### TIL and Tumor Cell Co-Culture

To perform the T cell and tumor cell co-culture assay, TILs with minimal in vitro culture (i.e., “Fresh” TILs) were thawed into T cell medium centrifuged and plated at 2×10⁶ cells/well in a 24-well plate in the absence of cytokines. After resting overnight at 37° C. and 5% CO₂, TIL (1×10⁵ cells) were cultured with or without autologous tumor cell line (1×10⁵ cells) in 200 ul of tumor cell media (RPMI+10% FBS) in a 96 well round-bottom plate. TILs were washed, counted, and resuspended in tumor cell media (RPMI+10% FBS) and were plated (1×10⁵ cells per well) in 100 ul of tumor cell media (RPMI+10% FBS) in a 96 well round-bottom plate. Allogenic and autologous tumor cell lines were washed once with PBS and were trypsinized to remove the cells from the plate. The tumor cells lines were then spun down and counted. The T cells were then incubated alone, or with 1×10⁵ of the allogenic and autologous tumor cell lines in 200 ul of culture volume with an E/T ratio of 1:1. Cells were cultured for 16 hrs. Cells were then harvested and sorted using flow cytometry.

### TCRA and TCRB Analysis

Bulk TILs, sorted T cells, or iPSCs at about 1.0×10⁵ cells per group were spun down, washed with 1×PBS and snap frozen in 50-100 ul of 1M HEPES buffer. For TIL-iPSCs, cells were trypsinized, dissociated into single cell suspension and counted in automated cell counter machine. Approximately 1.0×10⁵ cells from 15-20 individual iPSC lines were mixed and pooled together into a 15 ml tubes (referred to as “master tubes”) on ice. Samples were spun down at 1500 RPM for 5 minutes, washed once with 1×PBS, and were snap frozen in 100 ul of 1 M HEPES buffer. The remaining colonies after pick-up were collected after trypsinization and frozen down. Genomic DNA extraction and Immunoseq TCRB survey sequencing were performed by Adaptive Biotechnologies. The results were analyzed using IMMUNOSEQ® analyzer (Adaptive Biotechnologies, Seattle, Wash.). For pooled samples the TCRs with more than 0.5% productive TCRs were considered to be true.

### Isolation of PBMC

25 ml buffy coat bags from healthy volunteers were obtained from NIH blood bank prior to lymphocyte separation. Peripheral blood mononuclear cells (PBMCs) were isolated using Ficoll-Paque PLUS (GE Healthcare Bio-sciences AB).

### In Vitro Activation of T Cells

Healthy donor PBMC were sorted into for subsets as Naïve, central memory, effector memory and EMRA and were stimulated in vitro with anti-CD3 anti-CD28 dynabeads (Gibco) in a ratio of cells to beads 1:1 ratio (according to manufacturer's instructions) in complete media (RPMI+10% human serum) in presence of 300 IU rhIL-2 for 4 days. At day 4, beads were separated by magnet, and these cells were washed once with PBS and counted using automated cell counting machine (countess) for normalization of cell number from each group prior to Sendai virus infection.

### Retroviral Transduction of TCR Genes

93 GP cells were transfected with 1.5 μg of retroviral plasmid DNA encoding each TCR cloned into PMSGV1 along with 0.7 μg of helper plasmid RD114 using 10 μL Lipofectamine 2000 (Invitrogen) in OptiMEM (Invitrogen) for 8 hrs. Medium was replaced 8 h after transfection and cells were incubated for a further 48 h in complete media. To capture the viral particles, retroviral supernatants were spun at 2,000×g for 2 h at 32° C. in 6-well non-tissue-culture-treated plates coated with Retronectin (Takara Bio). Healthy donor peripheral blood lymphocytes were used as donor T cells for transduction. T cells were activated using 50 ng/ml OKT3 (milteny)/ml of media for 48 hours and 2 million cells were added/per well for infection. Around 5-6 days after TCR transduction, TCR transduced T cells were assessed for antigen specificity.

### Example 1

In this Example, methods of reprogramming T cells obtained from peripheral blood and TILs were investigated.

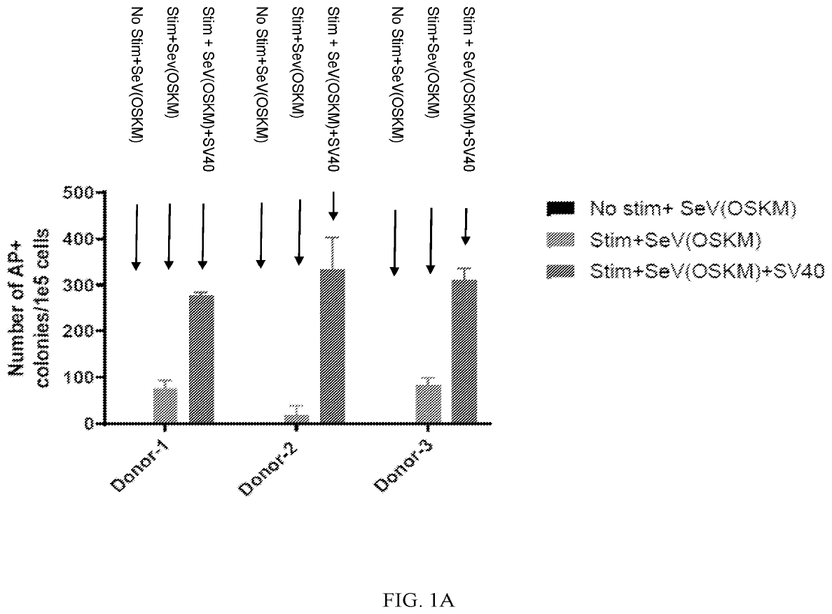

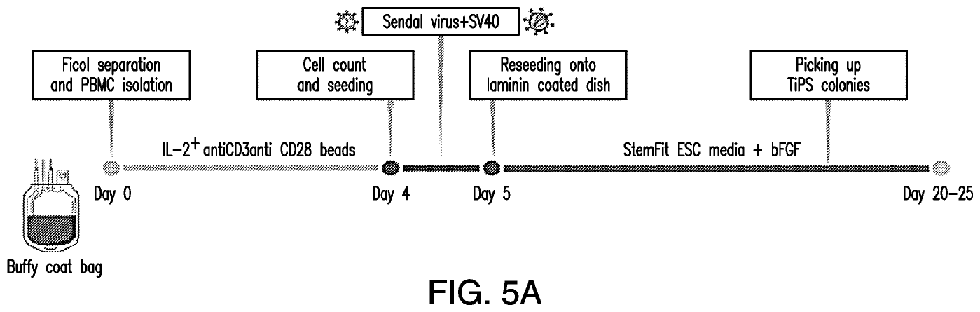

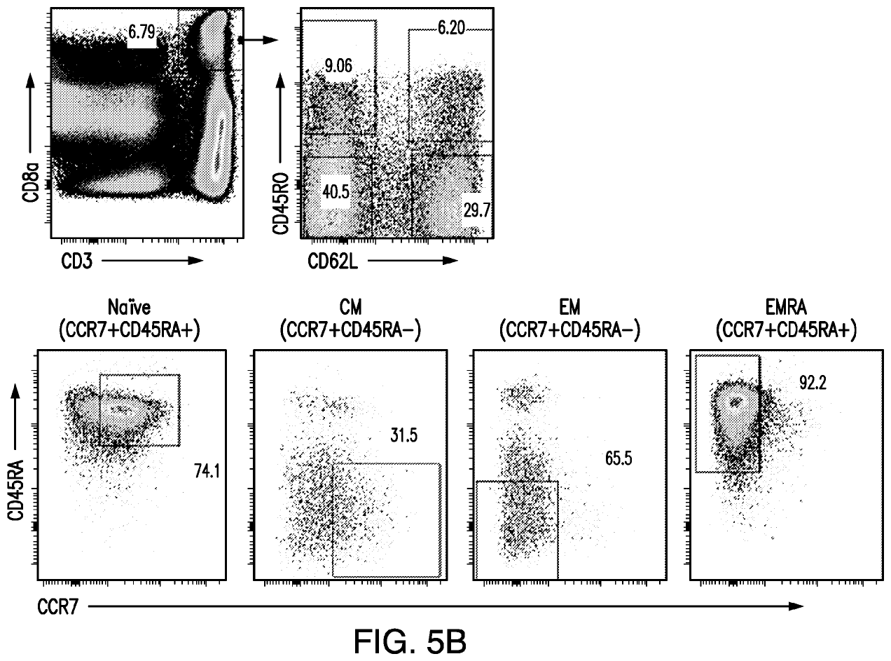

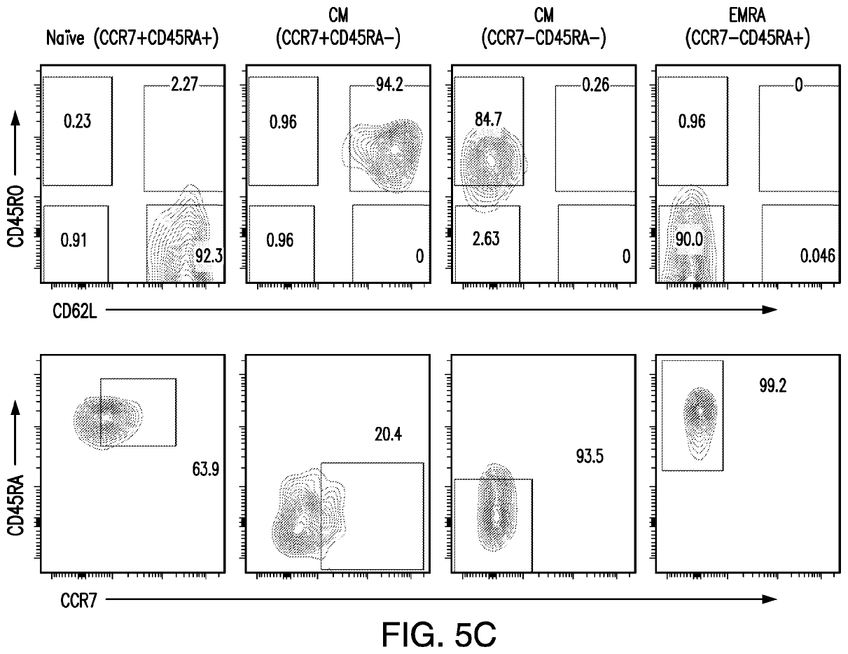

Peripheral blood T cells were reprogrammed into iPSCs with or without TCR stimulation by CD3/28 beads. (See FIG. 5A.) iPSCs were established only when the T cells were stimulated, and addition of SV40 enhanced the efficiency of reprogramming (See FIG. 1A). This indicates that TCR stimulation is a necessary step when reprogramming T cells. Appropriate stimulation conditions can generate iPSC from any T cell subset (i.e. naïve, CM, EM, EMRA). The applicable sorting strategy is illustrated by the representative FACS plots presented as FIGS. 5B and 5C.

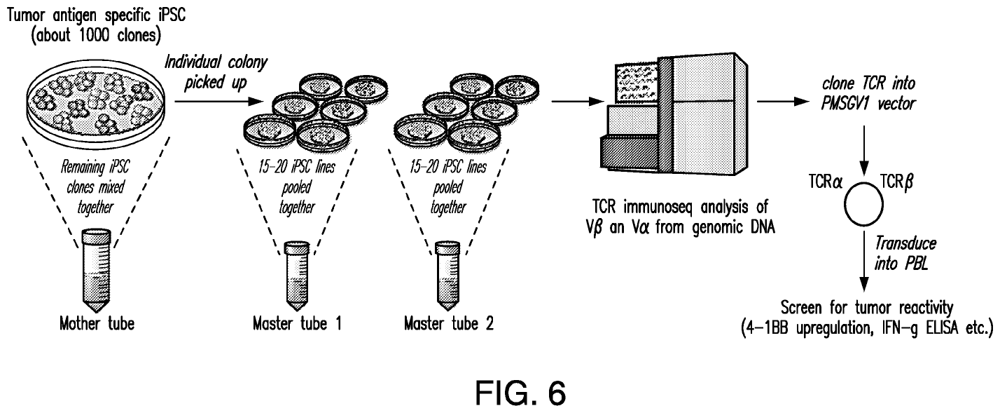

This approach was then used to reprogram tumor neoantigen specific T cells identified in a pancreatic cancer patient designated No. 4069. A diagram illustrating the general process for TCR sequencing of TIL-iPSCs and reactivity testing is provided in FIG. 6. Table 1 provides a summary table of the top ten TCR beta sequences in expanded TILs of patient 4069, indicating the CDR3 amino acid sequence, V beta family, and frequency in the bulk sample. The most frequent TCR was the patient's neoantigen specific TCR.

TABLE 1

| | | Frequency in |
|---|---|---|
| CDR3B | TCR VB family | TIL (%) |
| CASSLAGSKLYEQYF | TCRBV11-02*02 | 92.7 |
| (SEQ ID NO: 19) | | |
| CASRGKLNTEAFF | TCRBV05-01*01 | 1.07 |
| (SEQ ID NO: 20) | | |
| CASSLAGSKLYEQYF | TCRBV07-02*01 | 0.21 |
| (SEQ ID NO: 21) | | |
| CASSYEGGYNEQFF | TCRBV12 | 0.11 |
| (SEQ ID NO: 22) | | |
| CASSLAGSKLYEQYF | TCRBV11-03*01 | 0.07 |
| (SEQ ID NO: 23) | | |
| CASSIEVGEQYF | TCRBV19-01 | 0.07 |
| (SEQ ID NO: 24) | | |
| CASSLGQRGTIYF | TCRBV03 | 0.07 |
| (SEQ ID NO: 25) | | |
| CASSQGSGREIYEQYF | TCRBV14-01*01 | 0.06 |
| (SEQ ID NO: 26) | | |
| CASSEVPNIQYF | TCRBV02-01*01 | 0.06 |
| (SEQ ID NO: 27) | | |
| CASSEYPDRAGNTIYF | TCRBV10-02*01 | 0.05 |
| (SEQ ID NO: 28) | | |

As shown in Table 1, this population of TILs from patient 4069 was almost monoclonal with 92.7% expressing the TCR TCRBV11-02*02 (specific for the patient's neoantigen, ZFYVE27 R6H). This population of nearly monoclonal TIL were activated by CD3 antibodies, and were reprogrammed yielding TIL-iPSCs. Thirteen different TIL-iPSC clones were established and TCR beta genes were sequenced. Surprisingly, there were no TIL-iPSC clones with the TCRs specific for ZFYVE27 R6H (the patient's neoantigen) which made up 92.7% of the starting population; instead all of the TCRs were derived from a single T cell clone which was a very minor population that was not detected in the starting population by TCR deep sequencing. Table 2 is a summary table of TCR beta sequence of the single T cell clone indicating the CDR3 amino acid sequence, V beta family and the number of TIL-iPSC clones established using only an anti-CD3 antibody.

TABLE 2

| | TCR VB | # iPSC |
|---|---|---|
| CDR3B | family | clones |
| CASSQEVRTDNTEAFF | TCRBV04-02*01 | 13 |
| (SEQ ID NO: 29) | | |

Accordingly, this experiment demonstrates that while TCR stimulation is important for reprogramming T cells into iPSCs, it also shows that non-specific TCR stimulation by CD3 antibodies does not always generate TIL-iPSCs from tumor reactive T cells even from expanded TILs which were highly enriched for a tumor neoantigen specific clone. Because the frequency of tumor reactive T cells is very low in TIL, non-specific CD3 stimulation is not effective for selective reprogramming of tumor reactive T cells (even when applied to an expanded population of TILs highly enriched in tumor neoantigen specific clones).

Further, upon stimulation with anti-CD3 and anti-CD28 beads it was possible to stimulate rare antigen-specific T cell clones from the TILs from patient 4069 (see Table 3). Table 3 summarizes TCR immunoseq analysis of the 27 different TCRs obtained via the modified method of stimulation using anti-CD3 anti-CD28 beads for reprogramming. TCR 1 is the pre identified reactive TCR that was present in all master tubes, and TCRs 2-26 are minor clones that were also found in iPSC clones. The table provides a list of the TCRs, respective CDR3 beta amino acid sequence of each TCR, their V beta family and the minimum number of iPSC found in master tubes.

TABLE 3

| | | | Least number |
|---|---|---|---|
| | | | of iPSC |
| TCR | CDR3B | TCR VB Family | clones |
| 1 | CASSLAGSKLYEQYF | TCRBV11-02*02 | 8 |
| | (SEQ ID NO: 30) | | |
| 2 | CASNQGTSGYNEQFF | TCRBV11-01*01 | 8 |
| | (SEQ ID NO: 31) | | |
| 3 | CASSKGGRPGNTIYF | TCRBV19-01 | 8 |
| | (SEQ ID NO: 32 | | |
| 4 | CASSTTVETGGYTF | TCRBV19-01 | 5 |
| | (SEQ ID NO: 33) | | |
| 5 | CASSQPIHGPDQPQHF | TCRBV23-01*01 | 4 |
| | (SEQ ID NO: 34) | | |
| 6 | CASEQYF | TCRBV12 | 4 |
| | (SEQ ID NO: 35) | | |
| 7 | CASSPPSGGSGNTIYF | TCRBV18-01*01 | 4 |
| | (SEQ ID NO: 36) | | |
| 8 | CASSPGQGVSEQYF | TCRBV03 | 4 |
| | (SEQ ID NO: 37 | | |
| 9 | CASSYFGQETQYF | TCRBV06 | 4 |
| | (SEQ ID NO: 38) | | |
| 10 | CAWSRHPGGYTF | TCRBV30-01*01 | 3 |
| | (SEQ ID NO: 39 | | |
| 11 | CASSVNTGELFF | TCRBV09-01 | 3 |
| | (SEQ ID NO: 40) | | |
| 12 | CASFGVNTEAFF | TCRBV05-01*01 | 2 |
| | (SEQ ID NO: 41) | | |
| 13 | CASRASTSGSWEQYF | TCRBV06-05*01 | 2 |
| | (SEQ ID NO: 42) | | |
| 14 | CASSPRTSGGPHEQYF | TCRBV03 | 2 |
| | (SEQ ID NO: 43) | | |
| 15 | CSARVTGNRRETQYF | TCRBV20 | 2 |
| | (SEQ ID NO: 44) | | |
| 16 | CAWSVRPTGRSSTEAFF | TCRBV30-01*01 | 2 |
| | (SEQ ID NO: 45) | | |
| 17 | CASSLGVPPRNTIYF | TCRBV11-03*01 | 2 |
| | (SEQ ID NO: 46) | | |
| 18 | CASRFSSGGTDTQYF | TCRBV05-01*01 | 2 |
| | (SEQ ID NO: 47) | | |
| 19 | CASMRPTGGRGEKLFF | TCRBV02-01*01 | 1 |
| | (SEQ ID NO: 48) | | |
| 20 | CASSFRGEAFF | TCRBV12 | 1 |
| | (SEQ ID NO: 49) | | |
| 21 | CASRGKLNTEAFF | TCRBV05-01*01 | 1 |
| | (SEQ ID NO: 50) | | |
| 22 | CASSYAANQPQHF | TCRBV07-02*01 | 1 |
| | (SEQ ID NO: 51) | | |
| 23 | CASSQARTGAQPQHF | TCRBV04-02*01 | 1 |
| | (SEQ ID NO: 52) | | |
| 24 | CAWSRTRPNTGELFF | TCRBV30-01*01 | 1 |
| | (SEQ ID NO: 53) | | |
| 25 | CASRRTPRGDEQYF | TCRBV28-01*01 | 1 |
| | (SEQ ID NO: 54) | | |
| 26 | CASSLEAGQPQHF | TCRBV11-03*01 | 1 |
| | (SEQ ID NO: 55) | | |
| 27 | CASNIPGGNSPLHF | TCRBV06-06 | 1 |
| | (SEQ ID NO: 56) | | |

Additionally, it was confirmed that various cancer antigen dependent T cells from different cancers and patients may be reprogrammed into iPSCs as summarized in Table 4. Table 4 provides a summary of four patient's TIL samples that were used for reprogramming. Surgery branch patient ID, cancer type, tissue used for tumor and T cell harvest, clonality of T cells, numbers of iPSC colonies generated, numbers of total TCRs obtained from each patient and numbers of pre-identified tumor antigen reactive TCRs obtained in iPSC clones are shown.

TABLE 4

| | | | | | Different | Tumor |
|---|---|---|---|---|---|---|
| | | | | | TCR | reactive |
| | | | | IPSC | identified | TCR |
| | | | | lines | in | iden- |
| Pt. | Tumor | Cell | Clonal- | esta- | iPSC | tified |
| ID | Type | Source | ity | blished | clones | in iPSC |
| 4069 | Pan- | Infusion | Mostly | 96 | 27 | 1 |
| | creatic | Bag | mono- | | | |
| | | | clonal | | | |
| 1913 | Mela- | Fragment | Poly- | 221 | 9 | 3 |
| | noma | Culture | clonal | | | |
| 3784 | Mela- | Fragment | Poly- | 178 | 25 | 1+ |
| | noma | Culture | clonal | | | (3 new) |
| 3759 | Mela- | Fragment | Poly- | 330 | 22 | 0 |
| | noma | Culture | clonal | | | |

### Example 2

This Example describes experiments investigating whether tumor reactive T cells may be selectively reprogrammed into TIL-iPSCs upon co-culture with autologous tumor cells.

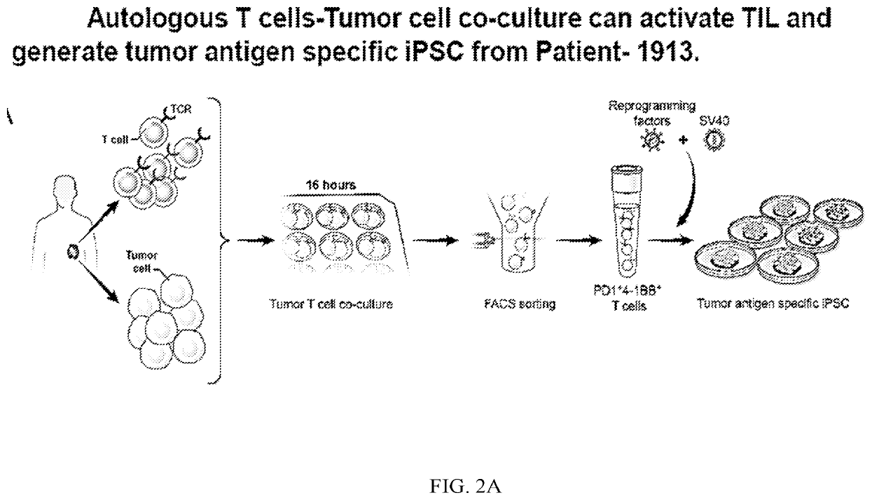

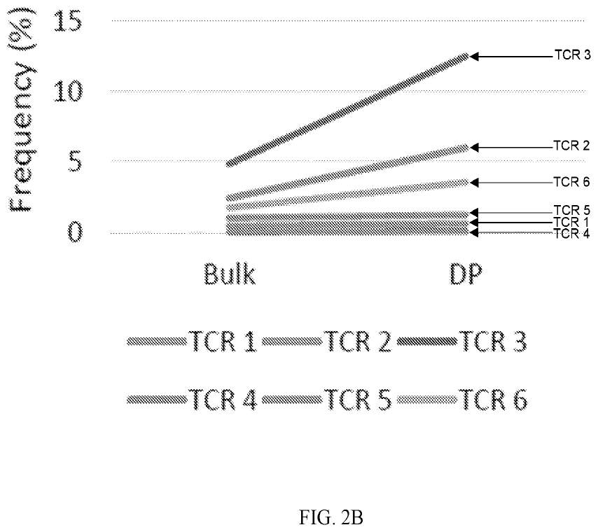

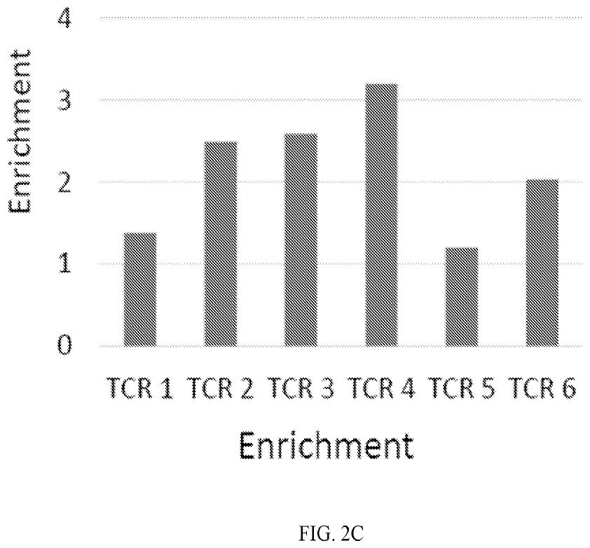

The method is summarized in FIG. 2A. For this study, TILs of melanoma patients were chosen according to the availability of TILs with minimal in vitro culture, the availability of autologous tumor cells and the presence of pre-identified tumor reactive TCRs.

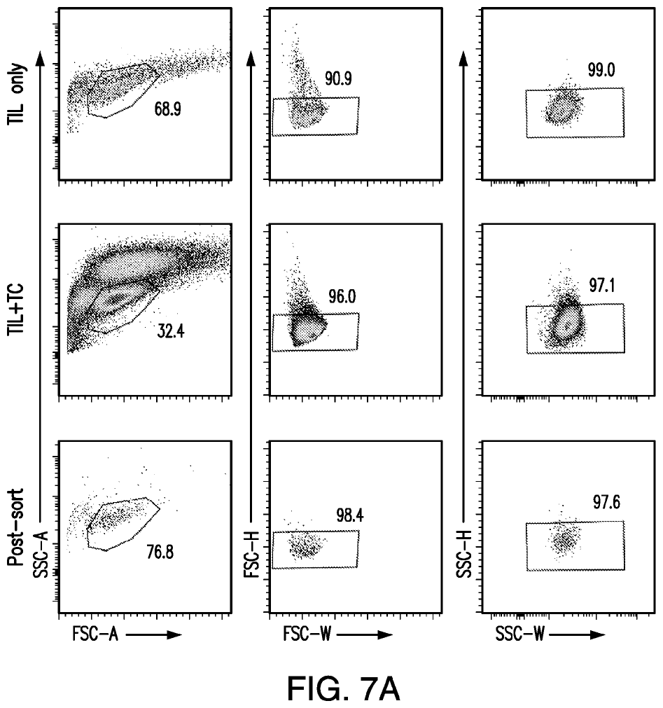

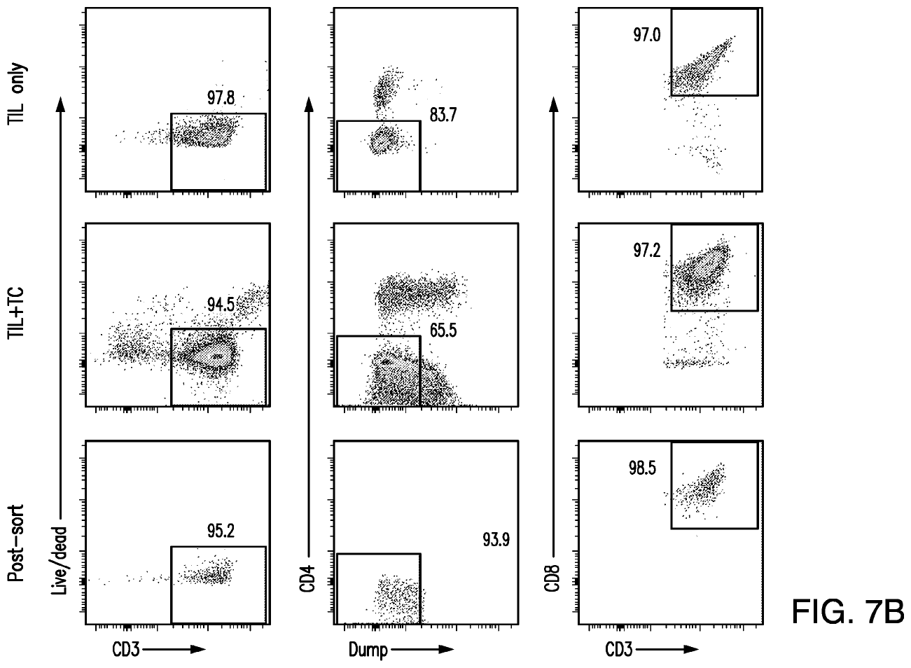

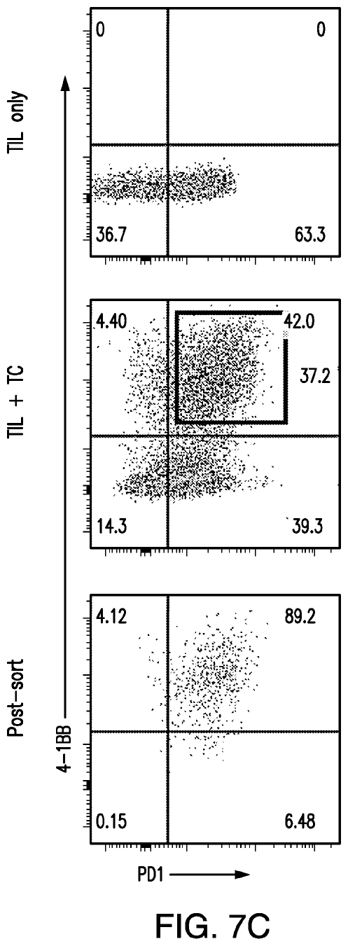

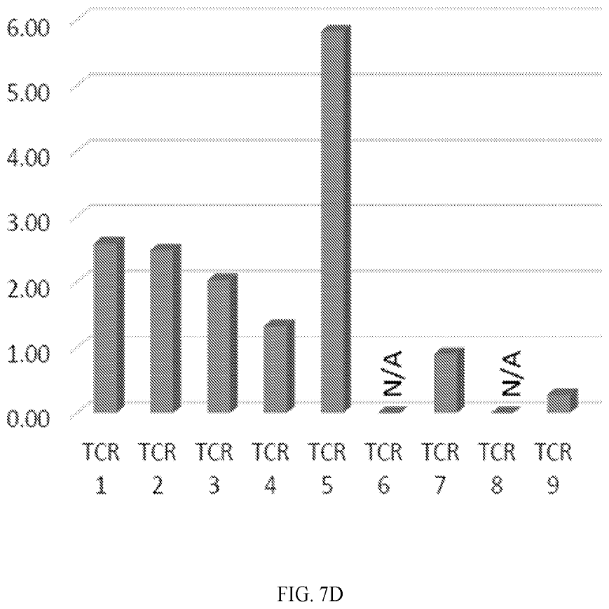

Specifically, the TILs from melanoma patient designated 1913 were selected. The fresh TILs were co-cultured with their autologous tumor cell line for 16 hours and CD3+CD4−CD8+PD1+ 4-1BB+ cells were sorted as shown in FIGS. 7A-C and infected with Sendai virus encoding the canonical Yamanaka factors and SV40 (i.e. reprogramming factors). (See FIGS. 2A and 7A-C). After three weeks, typical ES cell-like colonies appeared and 221 colonies were picked manually under microscope. The colonies remaining after picking the 221 were then pooled into a single sample. About 20 clones were pooled into each of 13 master tubes. Total DNA was extracted from each master tube, and TCRs were identified by TCR beta deep sequencing. In total nine different TCRs were detected from these 13 master tubes as summarized in Table 5. Table 5 summarizes TCR immunoseq analysis of the nine different TCRs obtained from the master tubes from patient 1913. From left to right, columns indicate the number of TCRs, respective CDR3 beta amino acid sequence of each TCR, their V beta family, frequency in bulk (before co-culture in starting material) and in sorted PD1+ 4-1BB+ (DP) population, enrichment (DP/Bulk) and presence of each TCR in established iPSC clones.

TABLE 5

| | | | Frequency % | | | Least number of |
|---|---|---|---|---|---|---|
| TCR | CDR3 beta | TCR VB family | Bulk | DP | Enrichment | iPSC clones |
| 1 | CASSKTSEFYEQYF | TCRBV07-08*01 | 4.83 | 12.5 | 2.59 | 13 |
| | (SEQ ID NO: 57) | | | | | |
| 2 | CASSLQGDLYEQYF | TCRBV07-08*01 | 2.41 | 5.99 | 2.49 | 13 |
| | (SEQ ID NO: 58) | | | | | |
| 3 | CASSVAISGEETQYF | TCRBV09-01 | 1.74 | 3.53 | 2.03 | 13 |
| | (SEQ ID NO: 59) | | | | | |
| 4 | CAWSETTAYEQYF | TCRBV30-01*01 | 15.0 | 20.0 | 1.33 | 13 |
| | (SEQ ID NO: 60) | | | | | |
| 5 | CASSFPNRPGNTIYF | TCRBV07-09 | 0.00372 | 0.0217 | 5.83 | 1 |
| | (SEQ ID NO: 61) | | | | | |
| 6 | CASSHMTSGRGSGEQYF | TCRBV03 | 0.0335 | n.d | N/A | 1 |
| | (SEQ ID NO: 62) | | | | | |
| 7 | CASSKQGRLLHF | TCRBV21-01*01 | 0.0112 | 0.0124 | 0.903 | 1 |
| | (SEQ ID NO: 63) | | | | | |
| 8 | CASSLGGTGDYEQYF | TCRBV06-05*01 | 0.0298 | n.d | N/A | 1 |
| | (SEQ ID NO: 64) | | | | | |
| 9 | CASSRPTSGAGDTQYF | TCRBV21-01*01 | 0.0112 | 0.00311 | 0.278 | 1 |
| | (SEQ ID NO: 65) | | | | | |

Four of the TCRs were detected in all 13 master tubes. The other five TCRs were each detected in only one of the master tubes. Six TCRs had been previously identified to be reactive against autologous tumor cells (Pasetto et al., Cancer Immunol Res; 4(9) September 2016) and all six of them were detectable in the fresh TIL and sorted PD-1+4-1BB+ populations in varying frequencies. The frequency of the 9 different TCRs in starting cells (isolated TILs; also referred to as Bulk) and after 16 hrs co-culture with their autologous tumor cells and sorted based on PD1+4-1BB+(DP) is summarized in Table 5. A graph depicting enrichment (i.e. the ratio of DP/Bulk frequencies) is provided as FIG. 7E.

Table 6 is a summary table of the TCR beta sequence analysis of the six previously identified (pre-identified) tumor reactive TCRs indicating the CDR3 amino acid sequence, V beta family, frequency in bulk (before co-culture in starting material) and in sorted PD1+ 4-1BB+(DP) population, enrichment (DP/Bulk) and presence in established iPSC clones. With respect to sample 5, this TCR was not found in established iPSC clones but was detected in the pooled sample of the remaining colonies (those left after picking up the 221 iPSC cell colonies), indicating that this TCR was present in the activated T cell population and could have been detected in iPSC if more colonies had been screened.

TABLE 6

| | | | Frequency % | | | Present |
|---|---|---|---|---|---|---|
| TCR | CDR3 of TCRVB | TCRVB family | Bulk | DP | Enrichment | in iPSC |
| 1 | CASSSPGTGSWGYTF | TCRBV12 | 0.454 | 0.627 | 1.38 | no |
| | (SEQ ID NO: 66) | | | | | |
| 2 | CASSLQGDLYEQYF | TCRBV07-08*01 | 2.41 | 5.99 | 2.49 | yes |
| | (SEQ ID NO: 67) | | | | | |
| 3 | CASSKTSEFYEQYF | TCRBV07-08*01 | 4.83 | 12.5 | 2.59 | yes |
| | (SEQ ID NO: 68) | | | | | |
| 4 | CASSWTGSNYGYTF | TCRBV05-01*01 | 0.0223 | 0.0714 | 3.20 | no |
| | (SEQ ID NO: 69) | | | | | |
| 5 | CASSLIMGLGSEQYF | TCRBV07-02*01 | 1.03 | 1.24 | 1.20 | no* |
| | (SEQ ID NO: 70) | | | | | |
| 6 | CASSVAISGEETQYF | TCRBV09-01 | 1.74 | 3.53 | 2.03 | yes |
| | (SEQ ID NO: 71) | | | | | |

The frequency of the six pre-identified TCRs was higher in the PD-1+4-1BB+ populations compared to the starting bulk population, showing that the enrichment of PD-1+4-1BB+ populations before reprogramming is an effective strategy. (See FIGS. 2C and 2D.) Three out of the six pre-identified tumor antigen specific TCRs were present in relatively high frequency (>2%) in the PD-1+4-1BB+ populations. These TCRs were detected in all iPSC master tubes suggesting that the TCRs were carried by a high number of clones (i.e. at least 13). The other three pre-identified tumor antigen specific TCRs were detected in the PD-1+4-1BB+ population with relatively low frequency (<2%) and were not detected in any of the iPSC master tubes.

Accordingly, this Example demonstrates the reprogramming of TILs to iPSCs by stimulating TILs with autologous tumor cells and enriching reactive populations identified based on PD-1 and 4-1BB expression.

### Example 3

This example describes the reprogramming of TILs to iPSCs, and subsequent analysis thereof.

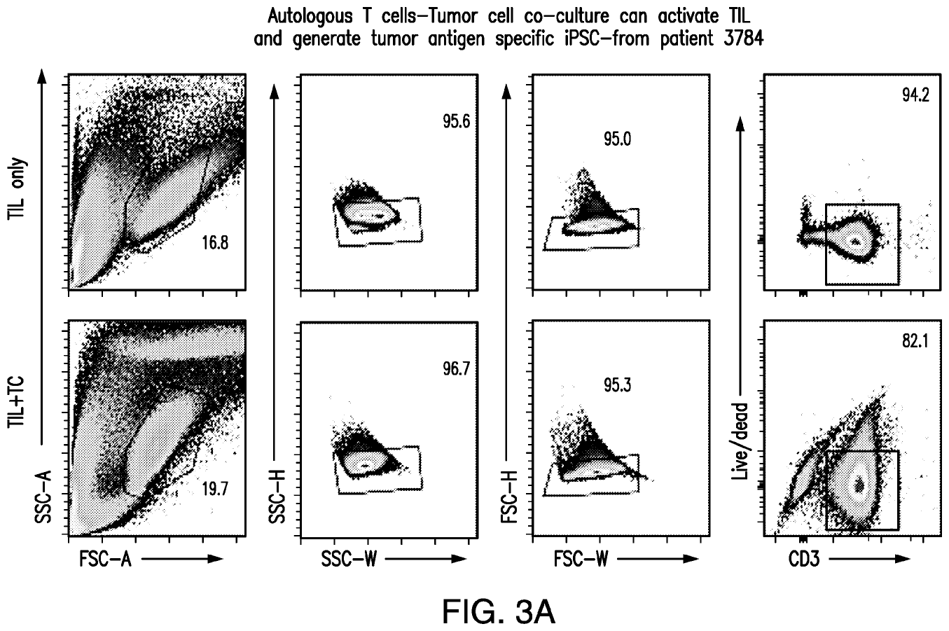

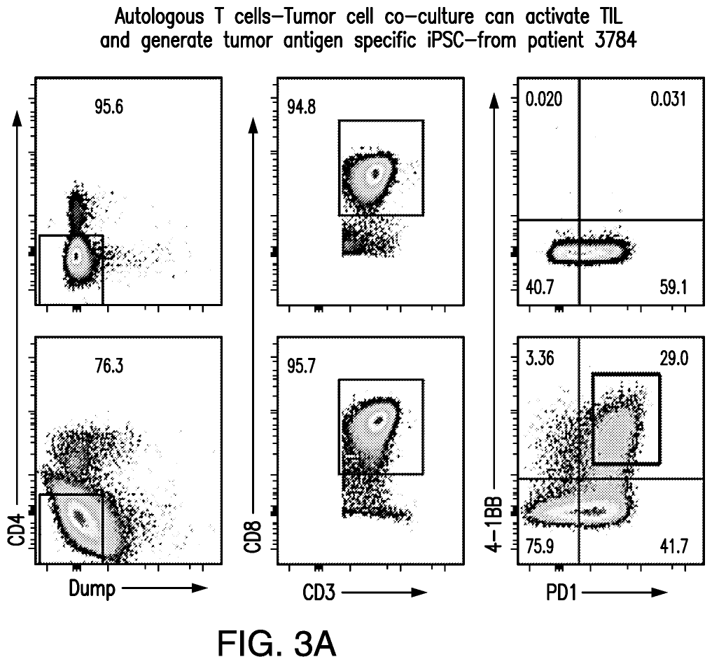

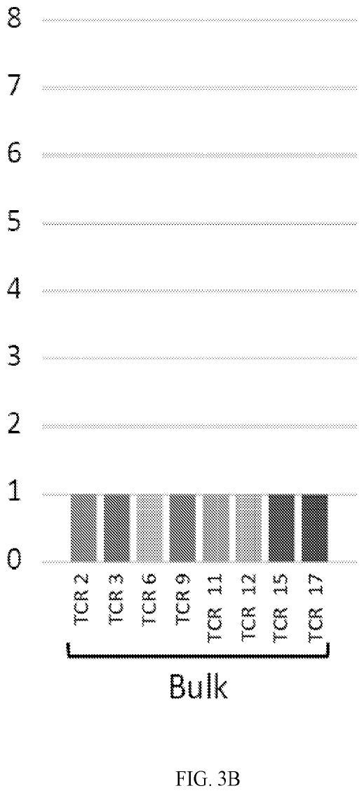

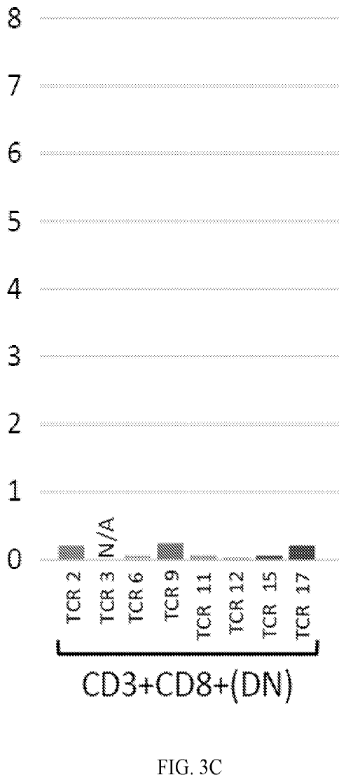

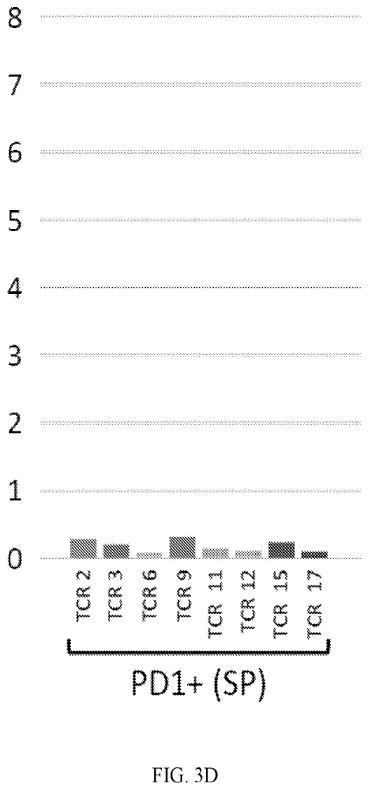

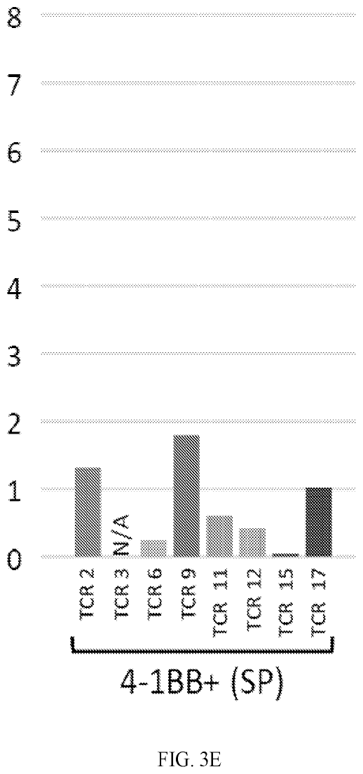

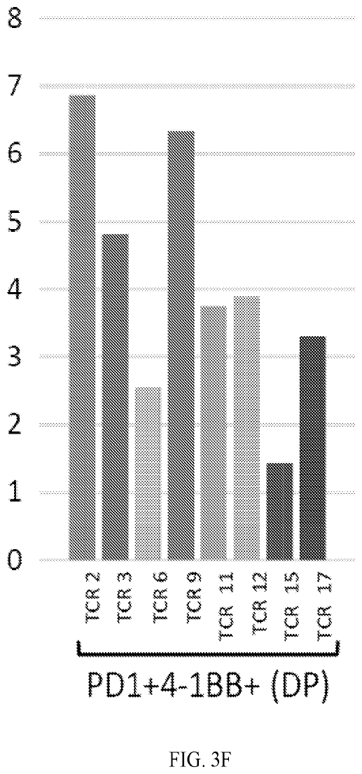

TILs from Patient #3784 were co-cultured with the autologous tumor cell line. CD3+CD4−CD8+PD1+ 4-1BB+ cells were sorted (See FIG. 3A) and reprogrammed as previously described into iPSCs. A total of 178 iPSC lines were established and TCR beta sequences were identified. Using various methods, eight different TCRs were pre-identified as specific for the mutated antigen expressed by the patient's tumor cells. The majority were relatively low frequency in the initial bulk TIL and PD-1+4-1BB+ populations. Table 7 provides a summary of the TCR beta sequence analysis of the eight pre-identified tumor reactive TCRs. Table 7 lists the CDR3 amino acid sequence, V beta family, frequency in bulk population, frequency in sorted PD1+ 4-1BB+(DP) population, enrichment (DP/Bulk) and presence in established iPSC clones.

TABLE 7

| | | Frequency % | | Enrichment | |
|---|---|---|---|---|---|
| CDR3b | TCRvB family | Bulk | DP | (DP/Bulk) | iPSC clone |
| CASSLVDRRGEKLFF | TCRBV07-06*01 | n.d | 0.00008 | N/A | no |
| (SEQ ID NO: 72) | | | | | |
| CASSRDRSNEQFF | TCRBV28-01*01 | 0.01 | 0.05 | 4.09 | no |
| (SEQ ID NO: 73) | | | | | |
| CAISALGQGSAYEQYF | TCRBV10-03*01 | 0.07 | 0.24 | 3.51 | no |
| (SEQ ID NO: 74) | | | | | |
| CASSASTGRSGNTIYF | TCRBV07-09 | 7.18 | 10.30 | 1.43 | 2 |
| (SEQ ID NO: 75) | | | | | |
| CASSRDGRVHQPQHF | TCRBV02-01*01 | 3.12 | 0.36 | 0.12 | no |
| (SEQ ID NO: 76) | | | | | |
| CASSLIRSEAFF | TCRBV28-01*01 | 0.61 | 0.55 | 0.90 | no |
| (SEQ ID NO: 77) | | | | | |
| CASKVMGQGSDNEQFF | TCRBV07-09 | 1.83 | 0.54 | 0.30 | no |
| (SEQ ID NO: 78) | | | | | |
| CASRARVSPLSGANVLTF | TCRBV06 | 0.04 | 0.03 | 0.82 | no |
| (SEQ ID NO: 79) | | | | | |

As can be seen in Table 7, one major TCR was 1.43 times more prevalent in the PD-1+4-1BB+ population as compared to bulk population, and was detected in two of nine master tubes. Others were not detected in established clones. A total of 25 different TCR beta chains were detected from 9 master tubes. Table 8 summarizes TCR immunoseq analysis of the 25 different TCRs obtained from patient 3784. From left to right, the columns indicate number of TCRs, respective CDR3 beta amino acid sequence of each TCR, their V beta family, frequency in bulk (before co-culture in starting material) and in the sorted PD1+ 4-1BB+ population (DP), enrichment (DP/Bulk) and presence of each TCR in established iPSC clones.

TABLE 8

| | | | Frequency % | | | Least number |
|---|---|---|---|---|---|---|
| TCR | CDR3B | TCRVB family | Bulk | DP | Enrichment | of iPSC clone |
| 1 | CASSEIGGSIYEQYF | TCRBV02-01*01 | n.d. | n.d. | N/A | 9 |
| | (SEQ ID NO: 80) | | | | | |
| 2 | CATSRSGAKNIQYF | TCRBV15-01*01 | 0.345 | 2.37 | 6.869 | 9 |
| | (SEQ ID NO: 81) | | | | | |
| 3 | CASNPRGRVYGYTF | TCRBV04-02*01 | 0.0328 | 0.158 | 4.817 | 8 |
| | (SEQ ID NO: 82) | | | | | |
| 4 | CASSATLGENIQYF | TCRBV07-09 | n.d. | n.d. | N/A | 7 |
| | (SEQ ID NO: 83) | | | | | |
| 5 | CASSLVAGYNEQFF | TCRBV07-09 | n.d. | n.d. | N/A | 7 |
| | (SEQ ID NO: 84) | | | | | |
| 6 | CASSVGGNPTYEQYF | TCRBV09-01 | 1.42 | 3.62 | 2.549 | 6 |
| | (SEQ ID NO: 85 | | | | | |
| 7 | CASSLSYGEQYF | TCRBV12 | 0.00117 | n.d. | N/A | 5 |
| | (SEQ ID NO: 86) | | | | | |
| 8 | CASSLSWDRVDGYTF | TCRBV27-01*01 | n.d. | n.d. | N/A | 4 |
| | (SEQ ID NO: 87) | | | | | |
| 9 | CASSQDSGLAGGQEFF | TCRBV04-03*01 | 0.0317 | 0.201 | 6.340 | 4 |
| | (SEQ ID NO: 88 | | | | | |
| 10 | CASSGGARDTDTQYF | TCRBV02-01*01 | n.d. | n.d. | N/A | 3 |
| | (SEQ ID NO: 89) | | | | | |
| 11 | CASSLAGGGEQYF | TCRBV05-01*01 | 4.19 | 15.7 | 3.747 | 3 |
| | (SEQ ID NO: 90) | | | | | |
| 12 | CASSPGTENTGELFF | TCRBV18-01*01 | 0.684 | 2.66 | 3.888 | 3 |
| | (SEQ ID NO: 91) | | | | | |
| 13 | CASSSDSGSHDNEQFF | TCRBV09-01 | n.d. | n.d. | N/A | 3 |
| | (SEQ ID NO: 92) | | | | | |
| 14 | CASSSGQGEYREQYF | TCRBV05-01*01 | n.d. | n.d. | N/A | 3 |
| | (SEQ ID NO: 93) | | | | | |
| 15 | CASSASTGRSGNTIYF | TCRBV07-09 | 7.18 | 10.3 | 1.434 | 2 |
| | (SEQ ID NO: 94) | | | | | |
| 16 | CASSEWRTGSNSPLHF | TCRBV02-01*01 | n.d. | n.d. | N/A | 2 |
| | (SEQ ID NO: 95) | | | | | |
| 17 | CASSLRASGRQETQYF | TCRBV27-01*01 | 0.669 | 2.21 | 3.303 | 2 |
| | (SEQ ID NO: 96) | | | | | |
| 18 | CASSSTGTGFNYGYTF | TCRBV07-09 | n.d. | n.d. | N/A | 2 |
| | (SEQ ID NO: 97) | | | | | |
| 19 | CASSTGYNQPQHF | TCRBV19-01 | 0.00352 | n.d. | N/A | 2 |
| | (SEQ ID NO: 98) | | | | | |
| 20 | CASSYSQVNIQYF | TCRBV27-01*01 | n.d. | n.d. | N/A | 2 |
| | (SEQ ID NO: 99) | | | | | |
| 21 | CASTSTPRGEQYF | TCRBV12 | n.d. | n.d. | N/A | 2 |
| | (SEQ ID NO: 100) | | | | | |
| 22 | CASSHIGRTYEQYF | TCRBV04-02*01 | n.d. | n.d. | N/A | 1 |
| | (SEQ ID NO: 101) | | | | | |
| 23 | CASSLADTTNTGELFF | TCRBV07-09 | n.d. | n.d. | N/A | 1 |
| | (SEQ ID NO: 102) | | | | | |
| 24 | CATSRLGLADYNEQFF | TCRBV15-01*01 | 0.00235 | n.d. | N/A | 1 |
| | (SEQ ID NO: 103) | | | | | |
| 25 | CSVGVRGGSYEQYF | TCRBV29-01*01 | n.d. | n.d. | N/A | 1 |
| | (SEQ ID NO: 104) | | | | | |

Interestingly, most of the TCRs identified in TIL-iPSC clones were undetectable or very low frequency in the initial TIL or PD-1+4-1BB+ populations. Some of them were highly enriched in PD-1+4-1BB+ population compared to bulk population. (See Table 8)

Without wishing to be bound by a particular theory, those low frequency T cell clones may have been activated by autologous tumor cells and preferentially reprogrammed to TIL-iPSCs.

To determine whether these TCRs are reactive to the patient's tumor cells, TCR alpha and beta chains from some of the TIL-iPSC lines were sequenced and identified. Table 9 summarizes four candidate TCR alpha and beta pairs identified in the TIL-iPSC population which were sequenced individually. The table includes CDR3 amino acid sequences of alpha and beta chains, VA family, TCR AJ family, VB, DB and JB family, and immunoseq analysis of TCR beta which contains frequency in bulk (before co-culture in starting material) and in sorted DP population, and enrichment (DP population/Bulk). Positive control is a pre-identified tumor cell reactive TCR (PIR-TCR) with relatively high frequency in the bulk TIL population (7.18%). To reduce pairing with endogenous TCR, a murine TCR constant region was used. Human constant region sequences available from public databases could also be used (see for example, UniProt P01848 (TRAC), P01850 (TRBC1), UniProtKB—A0A0G2JMB4 (TRBC2).

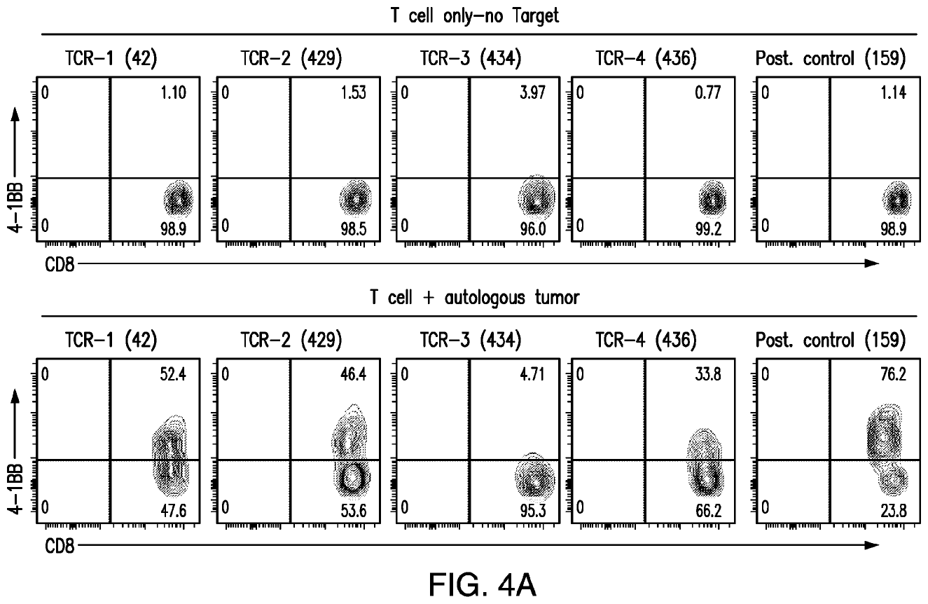

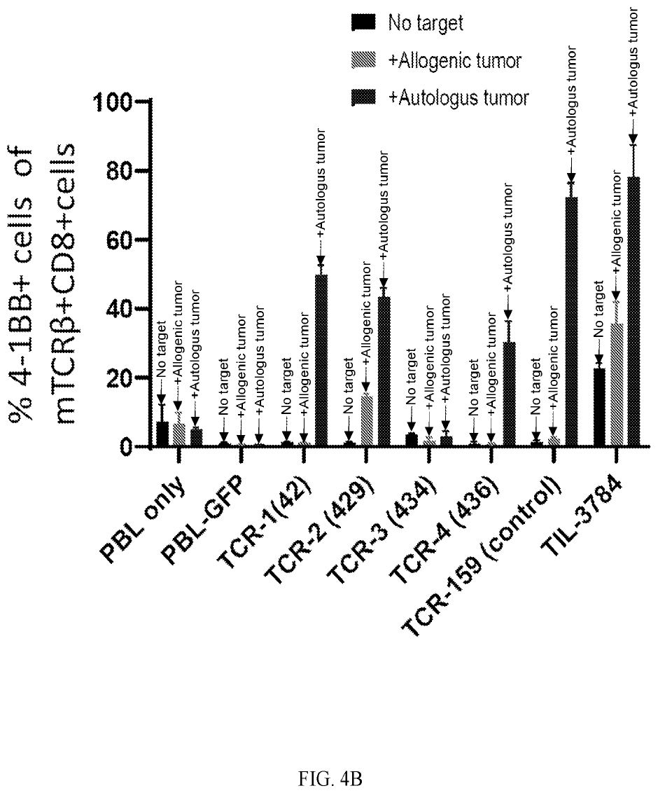

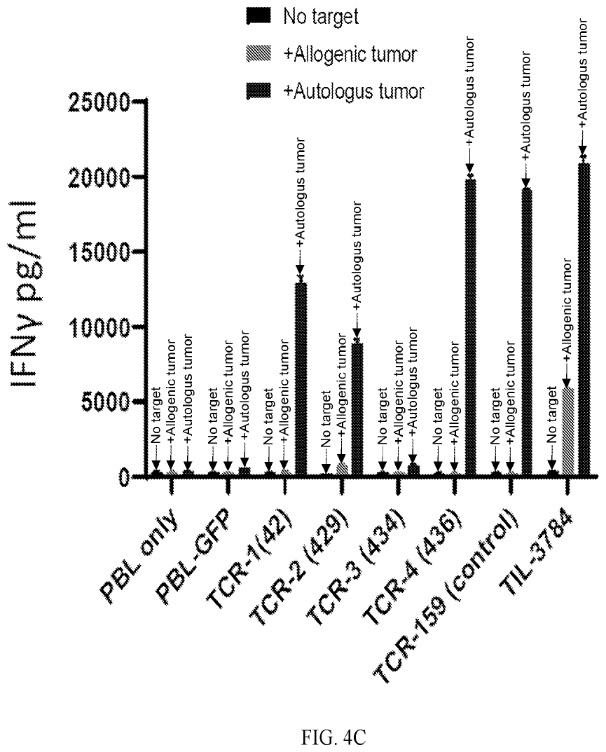

Three TCR pairs from TIL-iPSC clones whose TCR beta sequences were not detected in the PD-1+4-1BB+ population, one TCR pair from a clone whose TCR beta was very low frequency (0.13%), and the positive control were all tested for specificity against autologous tumor cells. TCR alpha and beta pairs were cloned into retrovirus vectors and transduced to healthy donors peripheral blood T cells and tested for specific recognition of autologous tumor cells. Three out of four newly tested TCR pairs (TCR-1, 2, and 4) were reactive against autologous tumor cells as was the pre-identified reactive TCR (Positive control) (See FIGS. 4B, C and D.)

TBALE 9

| TCR | CDR3 | TCR VA | TCR AJ | CDR3 | TCR VB | TCR DB | TCR JB | Frequency | | Enrichment |
|---|---|---|---|---|---|---|---|---|---|---|
| Pair | alpha | family | family | beta | family | family | family | Bulk | DP | DP/Bulk |
| 1 | CAVNTN | TRAV1- | TRAJ27* | CASSATL | TRBV7- | TRBD1* | TRBJ2- | n.d. | n.d. | n/a |
| | AGKSTF | 2*01 | 01 | GENIQYF | | 01 | 4*01 | | | |
| | (SEQ ID | | | (SEQ ID | 9*01 | | | | | |
| | NO: 105) | | | NO: 110) | | | | | | |
| 2 | CAHYNQ | TRAV1- | TRAJ23* | CASSLVA | TRBV7- | TRBD2* | TRBJ2- | n.d. | n.d. | n/a |
| | GGKLIF | 2*03 | 01 | GYNEQFF | | 01 | 1*01 | | | |
| | (SEQ ID | | | (SEQ ID | 9*01 | | | | | |
| | NO: 106) | | | NO: 111) | | | | | | |
| 3 | CAARAA | TRAV23/ | TRAJ17* | CASSLSY | TRBV12- | TRBD1* | TRBJ2- | 0.00117 | n.d | n/a |
| | GNKLTF | DV6*03 | 01 | GYEQYF | | 01 | 7*01 | | | |
| | (SEQ ID | | | (SEQ ID | 3*01 | | | | | |
| | NO: 107) | | | NO: 112) | | | | | | |
| 4 | CALTLE | TRAV9- | TRAJ54* | CASNPRG | TRBV4- | TRBD2* | TRBJ1- | 0.03 | 0.16 | 4.82 |
| | GAQKLVF | 2*01 | 01 | RVYGYTF | | 01 | 2*01 | | | |
| | (SEQ ID | | | (SEQ ID | 2*01 | | | | | |
| | NO: 108) | | | NO: 113) | | | | | | |
| Pos | CAVYTN | TRAV1-1*01 | TRAJ27* | CASSASTG | TRBV7- | TRBD2* | TRBJ1- | 7.18 | 10.3 | 1.43 |
| Ctrl | AGKSTF | | 01 | RSGNTIYF | | 01 | 3*01 | | | |
| | (SEQ ID | | | (SEQ ID | 9*01 | | | | | |
| | NO: 109) | | | NO: 114) | | | | | | |

Accordingly, this disclosure demonstrates that reprogramming of TILs to iPSCs by stimulating TILs with autologous tumor cells and enriching reactive populations identified based on PD-1 and 4-1BB expression can efficiently identify rare tumor antigen specific TCRs. The frequency of tumor specific T cells in starting TIL population has been an important factor for successful cloning of tumor antigen specific T cells. Even using the most advanced technology to identify TCR alpha and beta chains from single cells, the detection limit is still about one in one thousand cells, in part due to throughput capacity. The method disclosed here detected tumor reactive TCRs that were undetectable by deep sequencing of bulk TIL or sorted PD-1+4-1BB+ populations. These tumor antigen-specific TCRs can be used to genetically modify patient PBMCs which can be used for adoptive cell therapy. Additionally, the present disclosure provides effective methods for generating iPSCs having a more diverse set of TCRs than previously possible. These iPSCs can be redifferentiated into T cells for use in cell therapy.

TABLE 10
Description of certain sequences:

| SEQ ID | | |
|---|---|---|
| NO | Description | Sequence* |
| 1 | TCR Pair 1 CDR3 alpha | CAVNTNAGKSTF |
| 2 | TCR Pair 2 CDR3 alpha | CAHYNQGGKLIF |
| 3 | TCR Pair 3 CDR3 alpha | CAARAAGNKLTF |
| 4 | TCR Pair 4 CDR3 alpha | CALTLEGAQKLVF |
| 5 | TCR Pair 1 CDR3 beta | CASSATLGENIQYF |
| 6 | TCR Pair 2 CDR3 beta | CASSLVAGYNEQFF |
| 7 | TCR Pair 3 CDR3 beta | CASSLSYGYEQYF |
| 8 | TCR Pair 4 CDR3 beta | CASNPRGRVYGYTF |
| 9 | TCR Pair 1 alpha chain (not | MWGVFLLYVSMKMGGTTGQNIDQPTEMTATEGAIVQI |
| | including constant region) | NCTYQTSGFNGLFWYQQHAGEAPTFLSYNVLDGLEEK |
| | | GRFSSFLSRSKGYSYLLLKELQMKDSASYLCAVNTNA |
| | | GKSTFGDGTTLTVKP |
| 10 | TCR Pair 2 alpha chain | MWGVFLLYVSMKMGGTTGQNIDQPTEMTATEGAIVQI |
| | (not including constant | NCTYQTSGFNGLFWYQQHAGEAPTFLSYNVLDGLEEK |
| | region) | GRFSSFLSRSKGYSYLLLKELQMKDSASYLCAHYNQG |
| | | GKLIFGQGTELSVKP |
| 11 | TCR Pair 3 alpha chain | MDKILGASFLVLWLQLCWVSGQQKEKSDQQQVKQSPQ |
| | | SLIVOKGGISIINCAYENTAFDYFPWYQQFPGKGPAL |
| | (not including constant | LIAIRPDVSEKKEGRFTISFNKSAKQFSLHIMDSQPG |
| | region) | DSATYFCAARAAGNKLTFGGGTRVLVKP |
| 12 | TCR Pair 4 alpha chain | MNYSPGLVSLILLLLGRTRGNSVTQMEGPVTLSEEAF |
| | (not including constant | LTINCTYTATGYPSLFWYVQYPGEGLQLLLKATKADD |
| | region) | KGSNKGFEATYRKETTSFHLEKGSVQVSDSAVYFCAL |
| | | TLEGAQKLVFGQGTRLTINP |
| 13 | TCR Pair 1 beta chain | MGTSLLCWMALCLLGADHADTGVSQNPRHKITKRGQN |
| | (not including constant | VTFRCDPISEHNRLYWYRQTLGQGPEFLTYFQNEAQL |
| | region) | EKSRLLSDRFSAERPKGSFSTLEIQRTEQGDSAMYLC |
| | | ASSATLGENIQYFGAGTRLSVL |
| 14 | TCR Pair 2 beta chain | MGTSLLCWMALCLLGADHADTGVSQNPRHKITKRGQN |
| | (not including constant | VTFRCDPISEHNRLYWYRQTLGQGPEFLTYFQNEAQL |
| | region) | EKSRLLSDRFSAERPKGSFSTLEIQRTEQGDSAMYLC |
| | | ASSLVAGYNEQFFGPGTRLTVL |
| 15 | TCR Pair 3 beta chain | MDSWTFCCVSLCILVAKHTDAGVIQSPRHEVTEMGQE |
| | (not including constant | VTLRCKPISGHNSLFWYRQTMMRGLELLIYFNNNVPI |
| | region) | DDSGMPEDRFSAKMPNASFSTLKIQPSEPRDSAVYFC |
| | | ASSLSYGYEQYFGPGTRLTVT |
| 16 | TCR Pair 4 beta chain | MGCRLLCCAVLCLLGAVPMETGVTQTPRHLVMGMTNK |
| | (not including constant | KSLKCEQHLGHNAMYWYKQSAKKPLELMFVYNFKEQT |
| | region) | ENNSVPSRFSPECPNSSHLFLHLHTLQPEDSALYLCA |
| | | SNPRGRVYGYTFGSGTRLTVV |
| 17 | Murine TCR alpha constant | DIQNPEPAVYQLKDPRSQDSTLCLFTDFDSQINVPKT |
| | region | MESGTFITDKCVLDMKAMDSKSNGAIAWSNQTSFTCQ |
| | | DIFKETNATYPSSDVPCDATLTEKSFETDMNLNFQNL |
| | | LVIVLRILLLKVAGFNLLMTLRLWSS |
| 18 | Murine TCR beta constant | EDLRNVTPPKVSLFEPSKAEIANKOKATLVCLARGFF |
| | region | PDHVELSWWVNGKEVHSGVCTDPQAYKESNYSYCLSS |
| | | RLRVSATFWHNPRNHFRCQVQFHGLSEEDKWPEGSPK |
| | | PVTQNISAEAWGRADCGITSASYQQGVLSATILYEIL |
| | | LGKATLYAVLVSTLVVMAMVKRKNS |

*CDR3 underlined; murine constant domain italicized

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing an isolated population of tumor antigen specific T-cell induced pluripotent stem cells (T-iPSCs), the method comprising:
(a) isolating T cells from a first sample from a subject, wherein said subject has cancer;
(b) contacting the isolated T-cells of (a) with one or more tumor antigens to produce co-cultured T-cells;
(c) isolating from the co-cultured T-cells, T-cells expressing one or more T cell activation markers; and
(d) contacting the isolated T-cells of (c) with one or more reprogramming factors under conditions sufficient to reprogram the cells into T-iPSCs.

2. The method of claim 1, wherein the first sample is a tumor sample or PBMC.

3. The method of claim 1, wherein the isolated T cells of step (a) are tumor infiltrating lymphocytes.

4-15. (canceled)

16. The method of claim 1, wherein the one or more T cell activation marker(s) includes PD-1 or 4-1BB.

17. The method according to claim 1, wherein the reprogramming factors comprise one or more of: Kruppel-like factor 4 (Klf4), Sry-related HMG-box gene 2 (Sox2), Octamer-binding transcription factor 3/4 (Oct3/4), MYC protooncogene (c-Myc)) and Large T Antigen (SV40).

18-20. (canceled)

21. The method according to claim 1, further comprising differentiating T-iPSCs in the isolated population of T-iPSCs into T lineage cells to obtain differentiated T lineage cells.

22. A method of producing a medicament for the treatment or prevention of cancer in a subject having cancer, the method comprising:
(a) producing an isolated population of T-iPSCs according to the method of claim 1;
(b) differentiating the T-iPSCs into T lineage cells, to obtain differentiated T lineage cells; and
(c) formulating the differentiated T lineage cells into a medicament for the treatment or prevention of cancer in the subject.

23. A method of identifying a cancer antigen-specific TCR, the method comprising:
(a) producing an isolated population of T-iPSCs according to the method of claim 1; and
(b) determining the nucleotide sequence encoding the cancer antigen-specific TCR by performing RNA or DNA sequencing of nucleic acids comprised in the isolated population of T-iPSCs.

24-25. (canceled)

26. A composition comprising the iPSCs T-iPSCs produced according to the method of claim 1 and a pharmaceutically acceptable carrier.

27. A composition comprising the differentiated T lineage cells produced according to the method of claim 21 and a pharmaceutically acceptable carrier.

28-33. (canceled)

34. A method of identifying a tumor antigen specific TCR, comprising:
(a) isolating T cells from a first sample from the subject, wherein said subject has cancer;
(b) contacting the isolated T-cells of (a) with one or more tumor antigens to produce co-cultured T-cells;
(c) isolating from the co-cultured T-cells of (b) T-cells expressing one or more T cell activation markers;
(d) contacting the isolated T-cells of (c) with one or more reprogramming factors under conditions sufficient to reprogram the cells into T-iPSCs; and
(e) determining the DNA sequence encoding the TCR alpha and TCR beta chain from an iPSC colony.

35. The method of claim 34, further comprising the steps of
(f) transducing a peripheral blood mononuclear cells (PBMCs) with an expression vector comprising the sequence of the TCR alpha and TCR beta chains of (e); and
(g) contacting the transduced PBMCs with one or more tumor antigens associated with the cancer; and
(h) measuring reactivity of the transduced PBMCs to the one or more tumor antigens; wherein reactivity confirms that the TCR is tumor antigen-specific.

36-57. (canceled)

58. The method of claim 34 wherein the tumor antigen specific TCR represents less than 9%, 8%, 7%, 6%, 5%, 4%, 3%, 2%, 1%, or 0.1% of the isolated T-cells from the first sample from the subject.

59-60. (canceled)

61. A method of generating a polyclonal population of tumor antigen specific iPSC derived T cells, comprising:
(a) isolating T cells from a first sample from the subject, wherein said subject has cancer;
(b) contacting the isolated T-cells of (a) with one or more tumor antigens to produce co-cultured T-cells;
(c) isolating from the co-cultured T-cells of (b) T-cells expressing one or more T cell activation markers;
(d) contacting the isolated T-cells of (c) with one or more reprogramming factors under conditions sufficient to reprogram the cells into T-iPSCs; and
(e) differentiating the T-iPSCs into T lineage cells, to obtain differentiated T lineage cells.

62-80. (canceled)

81. A nucleic acid sequence encoding an amino acid sequence selected from SEQ ID NOs 1-16.

82. A recombinant TCR comprising:
(a) a CDR3 Va region of any one of SEQ ID Nos 1-4 and a CDR3 Vb region of any one of SEQ ID NOs. 5-8;
(b) A V alpha chain of SEQ ID NO:9 and a V beta chain of SEQ ID NO:13;
(c) A V alpha chain of SEQ ID NO:10 and a V beta chain of SEQ ID NO:14;
(d) V alpha chain of SEQ ID NO:11 and a V beta chain of SEQ ID NO:15; or
(e) V alpha chain of SEQ ID NO:12 and a V beta chain of SEQ ID NO:16.

83. A chimeric TCR comprising:
(a) a CDR3 Va region of any one of SEQ ID Nos 1-4 and a CDR3 Vb region of any one of SEQ ID NOs. 5-8;
(b) A V alpha chain of SEQ ID NO:9 and a V beta chain of SEQ ID NO:13;
(c) A V alpha chain of SEQ ID NO:10 and a V beta chain of SEQ ID NO:14;
(d) V alpha chain of SEQ ID NO:11 and a V beta chain of SEQ ID NO:15; or
(e) V alpha chain of SEQ ID NO:12 and a V beta chain of SEQ ID NO:16.

84. An isolated cell expressing a recombinant TCR of claim 82.

85-87. (canceled)

88. A method of treating or preventing cancer in a subject having cancer, the method comprising:
producing an isolated population of tumor antigen specific T-cell induced pluripotent stem cells (T-iPSCs) according to the method of claim 1; and
administering the isolated population of T-iPSCs to the subject in an amount effective to treat or prevent cancer in the subject.

89. A method of treating or preventing cancer in a subject having cancer, the method comprising:
producing T-lineage cells according to the method of claim 21; and
administering the T-lineage cells to the subject in an amount effective to treat or prevent cancer in the subject.


SEQUENCE LISTING (114 sequences)

SEQ ID NO: 1 - protein, 12 aa, Homo sapiens
CAVNTNAGKSTF

SEQ ID NO: 2 - protein, 12 aa, Homo sapiens
CAHYNQGGKLIF

SEQ ID NO: 3 - protein, 12 aa, Homo sapiens
CAARAAGNKLTF

SEQ ID NO: 4 - protein, 13 aa, Homo sapiens
CALTLEGAQKLVF

SEQ ID NO: 5 - protein, 14 aa, Homo sapiens
CASSATLGENIQYF

SEQ ID NO: 6 - protein, 14 aa, Homo sapiens
CASSLVAGYNEQFF

SEQ ID NO: 7 - protein, 13 aa, Homo sapiens
CASSLSYGYEQYF

SEQ ID NO: 8 - protein, 14 aa, Homo sapiens
CASNPRGRVYGYTF

SEQ ID NO: 9 - protein, 126 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVNTNAGKSTFGDGTTLTVKP

SEQ ID NO: 10 - protein, 126 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAHYNQGGKLIFGQGTELSVKP

SEQ ID NO: 11 - protein, 139 aa, Homo sapiens
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAARAAGNKLTFGGGTRVLVKP

SEQ ID NO: 12 - protein, 131 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALTLEGAQKLVFGQGTRLTINP

SEQ ID NO: 13 - protein, 133 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSATLGENIQYFGAGTRLSVL

SEQ ID NO: 14 - protein, 133 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLVAGYNEQFFGPGTRLTVL

SEQ ID NO: 15 - protein, 132 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLSYGYEQYFGPGTRLTVT

SEQ ID NO: 16 - protein, 132 aa, Homo sapiens
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASNPRGRVYGYTFGSGTRLTVV

SEQ ID NO: 17 - protein, 137 aa, Murine
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 18 - protein, 173 aa, Murine
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 19 - protein, 15 aa, Homo sapiens
CASSLAGSKLYEQYF

SEQ ID NO: 20 - protein, 13 aa, Homo sapiens
CASRGKLNTEAFF

SEQ ID NO: 21 - protein, 15 aa, Homo sapiens
CASSLAGSKLYEQYF

SEQ ID NO: 22 - protein, 14 aa, Homo sapiens
CASSYEGGYNEQFF

SEQ ID NO: 23 - protein, 15 aa, Homo sapiens
CASSLAGSKLYEQYF

SEQ ID NO: 24 - protein, 12 aa, Homo sapiens
CASSIEVGEQYF

SEQ ID NO: 25 - protein, 13 aa, Homo sapiens
CASSLGQRGTIYF

SEQ ID NO: 26 - protein, 16 aa, Homo sapiens
CASSQGSGREIYEQYF

SEQ ID NO: 27 - protein, 12 aa, Homo sapiens
CASSEVPNIQYF

SEQ ID NO: 28 - protein, 16 aa, Homo sapiens
CASSEYPDRAGNTIYF

SEQ ID NO: 29 - protein, 16 aa, Homo sapiens
CASSQEVRTDNTEAFF

SEQ ID NO: 30 - protein, 15 aa, Homo sapiens
CASSLAGSKLYEQYF

SEQ ID NO: 31 - protein, 15 aa, Homo sapiens
CASNQGTSGYNEQFF

SEQ ID NO: 32 - protein, 15 aa, Homo sapiens
CASSKGGRPGNTIYF

SEQ ID NO: 33 - protein, 14 aa, Homo sapiens
CASSTTVETGGYTF

SEQ ID NO: 34 - protein, 16 aa, Homo sapiens
CASSQPIHGPDQPQHF

SEQ ID NO: 35 - protein, 7 aa, Homo sapiens
CASEQYF

SEQ ID NO: 36 - protein, 16 aa, Homo sapiens
CASSPPSGGSGNTIYF

SEQ ID NO: 37 - protein, 14 aa, Homo sapiens
CASSPGQGVSEQYF

SEQ ID NO: 38 - protein, 13 aa, Homo sapiens
CASSYFGQETQYF

SEQ ID NO: 39 - protein, 12 aa, Homo sapiens
CAWSRHPGGYTF

SEQ ID NO: 40 - protein, 12 aa, Homo sapiens
CASSVNTGELFF

SEQ ID NO: 41 - protein, 12 aa, Homo sapiens
CASFGVNTEAFF

SEQ ID NO: 42 - protein, 15 aa, Homo sapiens
CASRASTSGSWEQYF

SEQ ID NO: 43 - protein, 16 aa, Homo sapiens
CASSPRTSGGPHEQYF

SEQ ID NO: 44 - protein, 15 aa, Homo sapiens
CSARVTGNRRETQYF

SEQ ID NO: 45 - protein, 17 aa, Homo sapiens
CAWSVRPTGRSSTEAFF

SEQ ID NO: 46 - protein, 15 aa, Homo sapiens
CASSLGVPPRNTIYF

SEQ ID NO: 47 - protein, 15 aa, Homo sapiens
CASRFSSGGTDTQYF

SEQ ID NO: 48 - protein, 16 aa, Homo sapiens
CASMRPTGGRGEKLFF

SEQ ID NO: 49 - protein, 11 aa, Homo sapiens
CASSFRGEAFF

SEQ ID NO: 50 - protein, 13 aa, Homo sapiens
CASRGKLNTEAFF

SEQ ID NO: 51 - protein, 13 aa, Homo sapiens
CASSYAANQPQHF

SEQ ID NO: 52 - protein, 15 aa, Homo sapiens
CASSQARTGAQPQHF

SEQ ID NO: 53 - protein, 15 aa, Homo sapiens
CAWSRTRPNTGELFF

SEQ ID NO: 54 - protein, 14 aa, Homo sapiens
CASRRTPRGDEQYF

SEQ ID NO: 55 - protein, 13 aa, Homo sapiens
CASSLEAGQPQHF

SEQ ID NO: 56 - protein, 14 aa, Homo sapiens
CASNIPGGNSPLHF

SEQ ID NO: 57 - protein, 14 aa, Homo sapiens
CASSKTSEFYEQYF

SEQ ID NO: 58 - protein, 14 aa, Homo sapiens
CASSLQGDLYEQYF

SEQ ID NO: 59 - protein, 15 aa, Homo sapiens
CASSVAISGEETQYF

SEQ ID NO: 60 - protein, 13 aa, Homo sapiens
CAWSETTAYEQYF

SEQ ID NO: 61 - protein, 15 aa, Homo sapiens
CASSFPNRPGNTIYF

SEQ ID NO: 62 - protein, 17 aa, Homo sapiens
CASSHMTSGRGSGEQYF

SEQ ID NO: 63 - protein, 12 aa, Homo sapiens
CASSKQGRLLHF

SEQ ID NO: 64 - protein, 15 aa, Homo sapiens
CASSLGGTGDYEQYF

SEQ ID NO: 65 - protein, 16 aa, Homo sapiens
CASSRPTSGAGDTQYF

SEQ ID NO: 66 - protein, 15 aa, Homo sapiens
CASSSPGTGSWGYTF

SEQ ID NO: 67 - protein, 14 aa, Homo sapiens
CASSLQGDLYEQYF

SEQ ID NO: 68 - protein, 14 aa, Homo sapiens
CASSKTSEFYEQYF

SEQ ID NO: 69 - protein, 14 aa, Homo sapiens
CASSWTGSNYGYTF

SEQ ID NO: 70 - protein, 15 aa, Homo sapiens
CASSLIMGLGSEQYF

SEQ ID NO: 71 - protein, 15 aa, Homo sapiens
CASSVAISGEETQYF

SEQ ID NO: 72 - protein, 15 aa, Homo sapiens
CASSLVDRRGEKLFF

SEQ ID NO: 73 - protein, 13 aa, Homo sapiens
CASSRDRSNEQFF

SEQ ID NO: 74 - protein, 16 aa, Homo sapiens
CAISALGQGSAYEQYF

SEQ ID NO: 75 - protein, 16 aa, Homo sapiens
CASSASTGRSGNTIYF

SEQ ID NO: 76 - protein, 15 aa, Homo sapiens
CASSRDGRVHQPQHF

SEQ ID NO: 77 - protein, 12 aa, Homo sapiens
CASSLIRSEAFF

SEQ ID NO: 78 - protein, 16 aa, Homo sapiens
CASKVMGQGSDNEQFF

SEQ ID NO: 79 - protein, 18 aa, Homo sapiens
CASRARVSPLSGANVLTF

SEQ ID NO: 80 - protein, 15 aa, Homo sapiens
CASSEIGGSIYEQYF

SEQ ID NO: 81 - protein, 14 aa, Homo sapiens
CATSRSGAKNIQYF

SEQ ID NO: 82 - protein, 14 aa, Homo sapiens
CASNPRGRVYGYTF

SEQ ID NO: 83 - protein, 14 aa, Homo sapiens
CASSATLGENIQYF

SEQ ID NO: 84 - protein, 14 aa, Homo sapiens
CASSLVAGYNEQFF

SEQ ID NO: 85 - protein, 15 aa, Homo sapiens
CASSVGGNPTYEQYF

SEQ ID NO: 86 - protein, 12 aa, Homo sapiens
CASSLSYGEQYF

SEQ ID NO: 87 - protein, 15 aa, Homo sapiens
CASSLSWDRVDGYTF

SEQ ID NO: 88 - protein, 16 aa, Homo sapiens
CASSQDSGLAGGQEFF

SEQ ID NO: 89 - protein, 15 aa, Homo sapiens
CASSGGARDTDTQYF

SEQ ID NO: 90 - protein, 13 aa, Homo sapiens
CASSLAGGGEQYF

SEQ ID NO: 91 - protein, 15 aa, Homo sapiens
CASSPGTENTGELFF

SEQ ID NO: 92 - protein, 16 aa, Homo sapiens
CASSSDSGSHDNEQFF

SEQ ID NO: 93 - protein, 15 aa, Homo sapiens
CASSSGQGEYREQYF

SEQ ID NO: 94 - protein, 16 aa, Homo sapiens
CASSASTGRSGNTIYF

SEQ ID NO: 95 - protein, 16 aa, Homo sapiens
CASSEWRTGSNSPLHF

SEQ ID NO: 96 - protein, 16 aa, Homo sapiens
CASSLRASGRQETQYF

SEQ ID NO: 97 - protein, 16 aa, Homo sapiens
CASSSTGTGFNYGYTF

SEQ ID NO: 98 - protein, 13 aa, Homo sapiens
CASSTGYNQPQHF

SEQ ID NO: 99 - protein, 13 aa, Homo sapiens
CASSYSQVNIQYF

SEQ ID NO: 100 - protein, 13 aa, Homo sapiens
CASTSTPRGEQYF

SEQ ID NO: 101 - protein, 14 aa, Homo sapiens
CASSHIGRTYEQYF

SEQ ID NO: 102 - protein, 16 aa, Homo sapiens
CASSLADTTNTGELFF

SEQ ID NO: 103 - protein, 16 aa, Homo sapiens
CATSRLGLADYNEQFF

SEQ ID NO: 104 - protein, 14 aa, Homo sapiens
CSVGVRGGSYEQYF

SEQ ID NO: 105 - protein, 12 aa, Homo sapiens
CAVNTNAGKSTF

SEQ ID NO: 106 - protein, 12 aa, Homo sapiens
CAHYNQGGKLIF

SEQ ID NO: 107 - protein, 12 aa, Homo sapiens
CAARAAGNKLTF

SEQ ID NO: 108 - protein, 13 aa, Homo sapiens
CALTLEGAQKLVF

SEQ ID NO: 109 - protein, 12 aa, Homo sapiens
CAVYTNAGKSTF

SEQ ID NO: 110 - protein, 14 aa, Homo sapiens
CASSATLGENIQYF

SEQ ID NO: 111 - protein, 14 aa, Homo sapiens
CASSLVAGYNEQFF

SEQ ID NO: 112 - protein, 13 aa, Homo sapiens
CASSLSYGYEQYF

SEQ ID NO: 113 - protein, 14 aa, Homo sapiens
CASNPRGRVYGYTF

SEQ ID NO: 114 - protein, 16 aa, Homo sapiens
CASSASTGRSGNTIYF